(19) United States
(12) Patent Application Publication
(10) Pub. No.: US 2026/0315557 A1
(43) Pub. Date: Oct. 15, 2026

(54) ONCOLYTIC VIRUS COMBINATION TO MAXIMIZE ONCOLYTIC ACTIVITY

(71) Applicant: Research Institute at Nationwide Children's Hospital, Columbus, OH (US)

(72) Inventor: Kevin A. Cassady, Columbus, OH (US)

(21) Appl. No.: 19/107,513

(22) PCT Filed: Jul. 7, 2023
(86) PCT No.: PCT/US2023/027164

§ 371 Date: Feb. 27, 2025

Related U.S. Application Data
(60) Provisional application No. 63/404,664, filed on Sep. 8, 2022.

(51) Int. Cl. A61K 35/768; A61K 35/26; A61K 35/763; A61K 38/00; A61P 35/00; C07K 14/47; C07K 14/54; C12N 5/078; C12N 7/00

(52) U.S. Cl. CPC A61K 35/768; A61K 35/26; A61K 35/763; A61P 35/00; C07K 14/4702; C07K 14/5434; C12N 5/0648; C12N 7/00; A61K 38/00; C12N 2510/00; C12N 2710/16632; C12N 2710/16643

(57) ABSTRACT

Provided are compositions comprising an oncolytic virus combination and methods of uses thereof.

### CROSS-REFERENCE TO RELATED PATENT APPLICATION

This application claims the benefit under 35 U.S.C. § 119(e) of U.S. Provisional Application No. 63/404,664, filed Sep. 8, 2022, the entire contents of which are incorporated herein by reference.

### STATEMENT OF GOVERNMENT SUPPORT

This invention was made with government support under CA232561 awarded by the National Institutes of Health and W81XWH-20-1-0340 awarded by the Dept. of Defense. The government has certain rights in the invention.

### BACKGROUND

Oncolytic viruses (OV) have been investigated in treating a cancer. However, what remains needed is an efficient therapy, the development of which is time and cost effective. The disclosure herein satisfies this need and provides related advantages as well.

### SUMMARY OF THE DISCLOSURE

In one aspect, provided herein is a composition comprising, or alternatively consisting essentially of, or yet further consisting of a first oncolytic virus expressing an anti-cancer cytokine, or an anti-cancer transcriptional regulator, or both; and a second oncolytic virus expressing one or more of: a cancer antigen, an immune cell engager specifically recognizing and binding to the cancer antigen, or an anti-cancer cytokine that is not the one expressed by the first oncolytic virus.

In another aspect, provided is a composition comprising, or alternatively consisting essentially of, or yet further consisting of an oncolytic virus expressing an anti-cancer cytokine, or an anti-cancer transcriptional regulator, or both; and an immune cell specifically recognizing and binding to a cancer antigen, with the proviso that (i) the anti-cancer cytokine does not comprise any one of interleukin 15 (IL-15), RANTES, interleukin 2 (IL-2), Tumor Necrosis Factor α (TNFα), or C-X-C Motif Chemokine Ligand 11 (CXCL11), or (ii) the immune cell does not comprise a Chimeric Antigen Receptor (CAR) T cell, or both (i) and (ii). In some embodiments, the anti-cancer cytokine comprises, or alternatively consists essentially of, or yet further consists of any one or more of: interleukin 12 (IL-12), interleukin 21 (IL-21), interleukin 27 (IL-27), or C-X-C Motif Chemokine Ligand 10 (CXCL10).

In yet another aspect, provided is an oncolytic virus expressing: an anti-cancer cytokine, or an anti-cancer transcriptional regulator, or both; and a cancer antigen, or an immune cell engager specifically recognizing and binding to the cancer antigen, or both. Also provided is a composition comprising, or alternatively consisting essentially of, or yet further consisting of an oncolytic virus as disclosed herein.

In one aspect, provided is a kit comprising, or alternatively consisting essentially of, or yet further consisting of instructions for use; a first composition comprising, or alternatively consisting essentially of, or yet further consisting of a first oncolytic virus expressing an anti-cancer cytokine, or an anti-cancer transcriptional regulator; and a second composition comprising, or alternatively consisting essentially of, or yet further consisting of a second oncolytic virus expressing one or more of: a cancer antigen, an immune cell engager specifically recognizing and binding the cancer antigen, or an anti-cancer cytokine that is not the one expressed by the first oncolytic virus.

Further provided is a method of treating a subject having a cancer. In some embodiments, the method comprises, or alternatively consists essentially of, or yet further consists of administering a composition as disclosed herein to the subject. In some embodiments, the method comprises, or alternatively consists essentially of, or yet further consists of administering the first composition and the second composition of a kit as described herein to the subject.

Additionally provided is a kit for use in a method as disclosed herein, comprising, or alternatively consisting essentially of, or yet further consisting of instructions for use and one or more of: a composition as disclosed herein, or an oncolytic virus as disclosed herein.

### BRIEF DESCRIPTION OF THE FIGURES

FIGS. 1A-1E shows tumor growth over time upon treatments of saline or PBS, C002 (C134 expressing IL-12) alone, C170 (C134 expressing EphA2) alone, and C170 plus C002 (noted as C170/C002). FIGS. 1A-1D plot sizes of the individual tumors in the mice treated by saline, C002, C170, and C170/C002, respectively. The tumor size changes were normalized to the initial tumor size, and the relative tumor growth data (i.e., average±S.E.M) from all four treatment groups is plotted in FIG. 1E. The treatment day was noted as Day 0.

FIGS. 2A-2B provide the survival curves of HSV naive mice treated with C002, HSV immunized mice treated with C002, and HSV immunized mice treated with C134, and show that virus-based cytokine expression (e.g., IL-12) improved immunotherapeutic response.

FIG. 2A compares HSV naive mice with HSV immunized mice, while FIG. 2B further adds mice treated with C134. More details are provided in Experimental Method No. 2.

FIGS. 3A-3E show that the expression levels of 502 genes significantly correlated with survival after G207 treatment. FIG. 3A provides an illustration of the G207 survival data, while FIG. 3B is a summary of the genes correlating with survival after treatment. Example of three of the genes (i.e., C-X-C motif chemokine 10, CXCL10, FIG. 3C; Receptor-type tyrosine-protein phosphatase epsilon, PTPRE, FIG. 3D, and Indoleamine 2,3-dioxygenase, IDO, FIG. 3E) analyzed and the relationship between gene expression and survival.

FIGS. 4A-4C provide that an ingenuity-based upstream regulatory analysis of the 502significant genes correlating with G207-survival show that these genes map (FIG. 4A) to immune response pathways (FIGS. 4B & 4C). These include both intrinsic antiviral response pathways (LEFT) and adaptive immune response pathways (including antigen presentation: RIGHT). As shown in FIG. 4A, Log2 Fold Change (best responder relative to worst responders), gene expression and activation Z score is represented as a heat map for the transcriptional regulators significantly associated with improved survival. FIG. 4B provides schematic representation of the significant upstream responses and the 5 cytokines of interest. Additionally, FIG. 4C shows the related pathways in a macrophage response.

### DETAILED DESCRIPTION

### Definitions

As it would be understood, the section or subsection headings as used herein is for organizational purposes only and are not to be construed as limiting and/or separating the subject matter described.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices, and materials are now described. All technical and patent publications cited herein are incorporated herein by reference in their entirety. Nothing herein is to be construed as an admission that the disclosure is not entitled to antedate such disclosure by virtue of prior disclosure.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach;

Harlow and Lane eds. (1999) Antibodies, A Laboratory Manual; Freshney (2005) Culture of Animal Cells: A Manual of Basic Techique, 5th edition, Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London);

Herzenberg et al. eds (1996) Weir's Handbook of Experimental Immunology; Manipulating the Mouse Embryo: A Laboratory Manual, 3rd edition (Cold Spring Harbor Laboratory Press (2002)); Sohail (ed.) (2004) Gene Silencing by RNA Interference: Technology and Application (CRC Press).

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compounds, agents, compositions and methods include the recited elements, but not exclude others. “Consisting essentially of” when used to define compounds, agents, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 20%, 10%, 5%, 1%, 0.5%, or even 0.1 % of the specified amount.

As used herein, comparative terms as used herein, such as high, low, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

As will be understood by one skilled in the art, for any and all purposes, all ranges disclosed herein also encompass any and all possible subranges and combinations of subranges thereof. Furthermore, as will be understood by one skilled in the art, a range includes each individual member.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The terms or “acceptable,” “effective,” or “sufficient” when used to describe the selection of any components, ranges, dose forms, etc. disclosed herein intend that said component, range, dose form, etc. is suitable for the disclosed purpose.

A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A composition as disclosed herein can be a pharmaceutical composition. A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

A combination as used herein intends that the individual active ingredients of the compositions are separately formulated for use in combination and can be separately packaged with or without specific dosages. The active ingredients of the combination can be administered concurrently or sequentially.

An “effective amount” is an amount sufficient to effect beneficial or desired results.

An effective amount can be administered in one or more administrations, applications, or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific agent employed, bioavailability of the agent, the route of administration, the age of the animal and its body weight, general health, sex, the diet of the animal, the time of administration, the rate of excretion, the drug combination, and the severity of the particular disorder being treated and form of administration. In general, one will desire to administer an amount of the agent that is effective to achieve a serum level commensurate with the concentrations found to be effective in vivo. These considerations, as well as effective formulations and administration procedures are well known in the art and are described in standard textbooks.

“Therapeutically effective amount” of an agent refers to an amount of the agent that is an amount sufficient to obtain a pharmacological response; or alternatively, is an amount of the agent that, when administered to a patient with a specified disorder or disease, is sufficient to have the intended effect, e.g., treatment, alleviation, amelioration, palliation or elimination of one or more manifestations of the specified disorder or disease in the patient.

A therapeutic effect does not necessarily occur by administration of one dose, and may occur only after administration of a series of doses. Thus, a therapeutically effective amount may be administered in one or more administrations.

As used herein, the phrase “derived from” means isolated from, purified from, or engineered from, or any combination thereof.

In some embodiments, the terms “first” “second” “third” “fourth” or similar in a component name are used to distinguish and identify more than one component sharing certain identity in their names. For example, “first oncolytic virus” and “second oncolytic virus” are used to distinguishing two oncolytic viruses.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

When the disease is cancer, the following clinical endpoints are non-limiting examples of treatment: (1) elimination of a cancer in a subject or in a tissue/organ of the subject or in a cancer loci; (2) reduction in tumor burden (such as number of cancer cells, number of cancer foci, number of cancer cells in a foci, size of a solid cancer, concentrate of a liquid cancer in the body fluid, and/or amount of cancer in the body); (3) stabilizing or delay or slowing or inhibition of cancer growth and/or development, including but not limited to, cancer cell growth and/or division, size growth of a solid tumor or a cancer loci, cancer progression, and/or metastasis (such as time to form a new metastasis, number of total metastases, size of a metastasis, as well as variety of the tissues/organs to house metastatic cells); (4) less risk of having a cancer growth and/or development; (5) inducing an immune response of the patient to the cancer, such as higher number of tumor-infiltrating immune cell, higher number of activated immune cells, or higher number cancer cell expressing an immunotherapy target, or higher level of expression of an immunotherapy target in a cancer cell; (6) higher probability of survival and/or increased duration of survival, such as increased overall survival (OS, which may be shown as 1-year, 2-year, 5-year, 10-year, or 20-year survival rate), increased progression free survival (PFS), increased disease free survival (DFS), increased time to tumor recurrence (TTR) and increased time to tumor progression (TTP). In some embodiments, the subject after treatment experiences one or more endpoints selected from tumor response, reduction in tumor size, reduction in tumor burden, increase in overall survival, increase in progression free survival, inhibiting metastasis, improvement of quality of life, minimization of drug-related toxicity, and avoidance of side-effects (e.g., decreased treatment emergent adverse events). In some embodiments, improvement of quality of life includes resolution or improvement of cancer-specific symptoms, such as but not limited to fatigue, pain, nausca/vomiting, lack of appetite, and constipation; improvement or maintenance of psychological well-being (e.g., degree of irritability, depression, memory loss, tension, and anxiety); improvement or maintenance of social well-being (e.g., decreased requirement for assistance with eating, dressing, or using the restroom; improvement or maintenance of ability to perform normal leisure activities, hobbies, or social activities; improvement or maintenance of relationships with family). In some embodiments, improved patient quality of life that is measured qualitatively through patient narratives or quantitatively using validated quality of life tools known to those skilled in the art, or a combination thereof. Additional non-limiting examples of endpoints include reduced hospital admissions, reduced drug use to treat side effects, longer periods off-treatment, and earlier return to work or caring responsibilities. In one aspect, prevention or prophylaxis is excluded from treatment.

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed of having or is suspected of having a cancer.

In one embodiment, the term “disease” or “disorder” as used herein refers to a cancer, a status of being diagnosed with a cancer, a status of being suspect of having a cancer, or a status of at high risk of having a cancer.

As used herein, a “cancer” is a disease state characterized by the presence in a subject of cells demonstrating abnormal uncontrolled replication and in some aspects, the term may be used interchangeably with the term “tumor.” It is a known medically as an uncontrolled division of abnormal cells in a part of the body, benign or malignant. In one embodiment, cancer refers to a malignant neoplasm, a broad group of diseases involving unregulated cell division and growth, and invasion to nearby parts of the body. Non-limiting examples of cancers include carcinomas, sarcomas, leukemia and lymphoma, e.g., colon cancer, colorectal cancer, rectal cancer, gastric cancer, esophageal cancer, head and neck cancer, breast cancer, brain cancer, lung cancer, stomach cancer, liver cancer, gall bladder cancer, or pancreatic cancer. In one embodiment, the term “cancer” refers to a solid tumor, which is an abnormal mass of tissue that usually does not contain cysts or liquid areas, including but not limited to, sarcomas, carcinomas, and certain lymphomas (such as Non-Hodgkin's lymphoma). In another embodiment, the term “cancer” refers to a liquid cancer, which is a cancer presenting in body fluids (such as, the blood and bone marrow), for example, leukemias (cancers of the blood) and certain lymphomas.

Additionally or alternatively, a cancer may refer to a local cancer (which is an invasive malignant cancer confined entirely to the organ or tissue where the cancer began), a metastatic cancer (referring to a cancer that spreads from its site of origin to another part of the body), a non-metastatic cancer, a primary cancer (a term used describing an initial cancer a subject experiences), a secondary cancer (referring to a metastasis from primary cancer or second cancer unrelated to the original cancer), an advanced cancer, an unresectable cancer, or a recurrent cancer. As used herein, an advanced cancer refers to a cancer that had progressed after receiving one or more of: the first line therapy, the second line therapy, or the third line therapy.

The term “cancer or tumor antigen” refers to an antigen known to be associated and expressed on the surface with a cancer cell or tumor cell or tissue.

A “solid tumor” is an abnormal mass of tissue that usually does not contain cysts or liquid areas. Solid tumors can be benign or malignant. Different types of solid tumors are named for the type of cells that form them. Examples of solid tumors include sarcomas, carcinomas, and lymphomas. In some embodiments, a solid tumor comprises bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, prostate cancer, or stomach cancer.

As used herein, the term “hematologic cancer” refers to cancers with hematopoietic origin. In some instances, the hematologic malignancy is a B-cell malignancy. In some instances, the hematologic malignancy is a lymphoma, optionally a B-cell lymphoma.

Exemplary hematologic malignancies include, but are not limited to, Diffuse large B-cell lymphoma (DLBCL), follicular lymphoma, chronic lymphocytic leukemia (CLL), small lymphocytic lymphoma (SLL), mantel cell lymphoma (MCL), marginal zone lymphomas, Burkitt lymphoma, Waldenström macroglobulinemia, hairy cell leukemia (HCL), primary central nervous system (CNS) lymphoma, or primary intraocular lymphoma.

The term “B-cell lymphoma or leukemia” refers to a type of cancer that forms in issues of the lymphatic system or bone marrow and has undergone a malignant transformation that makes the cells within the cancer pathological to the host organism with the ability to invade or spread to other parts of the body.

As used herein, the term “autologous,” in reference to cells refers to cells that are isolated and infused back into the same subject (recipient or host). “Allogeneic” refers to non-autologous cells.

As used herein, an ablative therapy is a treatment destroying or ablating cancer tumors. In one embodiment, the ablative therapy does not require invasive surgery. In other embodiments, the ablative therapy refers to removal of a tumor via surgery. In some embodiments, the step ablating the cancer includes immunotherapy of the cancer. Cancer immunotherapy is based on therapeutic interventions that aim to utilize the immune system to combat malignant diseases. It can be divided into unspecific approaches and specific approaches. Unspecific cancer immunotherapy aims at activating parts of the immune system generally, such as treatment with specific cytokines known to be effective in cancer immunotherapy (e.g. IL-2, interferon's, cytokine inducers).

The cancer cell to be treated can be a solid tumor or blood cancer, e.g., carcinoma or sarcoma and non-limiting examples of such include pancreatic cancer, renal cancer, small cell lung cancer, brain cancer, neuroblastoma, neural cancer, bone cancer, lymphoma, myeloma, colon cancer, uterine cancer, breast cancer, leukemia, liver cancer, prostate cancer, skin cancer, or melanoma. The cell is of any species, e.g., mammalian and human and when performed in vitro, it can be from a cultured cell line or a primary cell, e.g., from a tissue biopsy.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

“Administration” or “delivery” of an oncolytic virus or a composition containing same can be performed in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application. In some embodiments, the administration is administration to a tumor microenvironment. In some embodiments, administering or a grammatical variation thereof also refers to more than one doses with certain interval. In some embodiments, the interval is 1 day, 2 days, 3 days, 4 days, 5 days, 6 days, 1 week, 10 days, 2 weeks, 3 weeks, 1 month, 2 months, 3 months, 4 months, 5 months, 6 months, 1 year or longer. In some embodiments, one dose is repeated for once, twice, three times, four times, five times, six times, seven times, eight times, nine times, ten times or more.

The term administration shall include without limitation, administration by oral, parenteral (e.g., intramuscular, intraperitoneal, intravenous, intravascular, intraperitoneal, intracerebroventricular (ICV), intrathecal, intracisternal injection or infusion, intracranial, ocular, intradermally, percutaneously, subcutaneous injection, or implant), intratumorally, by inhalation spray nasal, intratracheal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The disclosure is not limited by the route of administration, the formulation or dosing schedule.

An agent of the present disclosure can be administered for therapy by any suitable route of administration. It will also be appreciated that the optimal route will vary with the condition and age of the recipient, and the disease being treated.

Administration or treatment in “combination” refers to administering two agents such that their pharmacological effects are manifest at the same time. Combination does not require administration at the same time or substantially the same time, although combination can include such administrations.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition”. In patients with cancer, primary treatment can be surgery, chemotherapy, radiation therapy, or a combination of these therapies. First line therapy is also referred to those skilled in the art as “primary therapy and primary treatment.” See National Cancer Institute website at www. cancer. gov, last visited on May 1, 2008. Typically, a patient is given a subsequent chemotherapy regimen because the patient did not show a positive clinical or sub-clinical response to the first line therapy or the first line therapy has stopped.

The terms “oligonucleotide” or “polynucleotide” or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

As used herein, the term “purified” does not require absolute purity; rather, it is intended as a relative term. Thus, for example, a purified nucleic acid, peptide, protein, biological complexes, cell, virus or other active compound is one that is isolated in whole or in part from proteins or other contaminants. Generally, substantially purified peptides, proteins, biological complexes, cell, virus or other active compounds for use within the disclosure comprise more than 80% of all macromolecular species present in a preparation prior to admixture or formulation of the peptide, protein, biological complex, cell, virus or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex, cell, virus or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

In some embodiments, the term “engineered” or “recombinant” refers to having at least one modification not normally found in a naturally occurring protein, polypeptide, polynucleotide, strain, wild-type strain or the parental host strain of the referenced species. In some embodiments, the term “engineered” or “recombinant” refers to being synthetized by human intervention.

The term “a regulatory sequence” “a regulatory element” “an expression control element” or “promoter” as used herein, intends a polynucleotide that is operatively linked to a polynucleotide to be transcribed and/or replicated, and facilitates the expression and/or replication of the polynucleotide. Non-limiting examples of a regulatory sequence include a promoter, an enhancer, or a polyadenylation sequence.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting examples of promoters include a cytomegalovirus CMV promoter or retroviral long terminal repeat (LTR) promoter. See, for example, Weber et al. Hum Gene Ther. 2007 September; 18(9):849-60.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

The term “express” refers to the production of a gene product, such as mRNA, peptides, polypeptides or proteins. As used herein, “expression” refers to the process by which polynucleotides are transcribed into mRNA or the process by which the transcribed mRNA is subsequently being translated into peptides, polypeptides, or proteins. If the polynucleotide is derived from genomic DNA, expression may include splicing of the mRNA in a eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In some embodiments, the vector is a virus (i.e., a viral vector or oncolytic viral vector).

As used herein, the term “vector” refers to a nucleic acid construct deigned for transfer between different hosts, including but not limited to a plasmid, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector.

The term “vector genome” refers to the nucleic acid component of a virus particle, which encodes the genome of the virus particle including any proteins required for replication and/or integration of the genome. In some embodiments, a viral genome acts as a viral vector and may comprise a heterologous gene operably linked to a regulatory sequence, such as a promoter. The promoter may be either native or heterologous to the gene and may be viral or non-viral in origin. The viral genomes described herein may be based on any virus, may be an RNA or DNA genome, and may be either single stranded or double stranded.

An “oncolytic virus” or “OV” refers to a virus that preferentially infects a cancer cell as compared to normal cells. In further embodiments, an oncolytic virus preferentially lyses a cancer cell as compared to normal cells. In some embodiments, an oncolytic virus refers to a virus that selectively replicates in a cancer cell, and thus lyses the cancer cell. In further embodiments, as the infected cancer cells are destroyed by oncolysis, they release new infectious virus particles or virions to help destroy the remaining cancer cells.

In some embodiments, the oncolytic virus is or is derived from adenovirus, adeno-associated virus (AAV), reovirus, measles, herpes simplex virus (HSV), Newcastle disease virus (NDV), vesicular stomatitis virus (VSV), poliovirus (PV), ECHO-7 (RIGVIR), semliki forest virus (SFV), maraba virus, coxsackievirus A21, senecavirus, a Pox virus, a retrovirus, a vesiculovirus, a parvovirus, or an influenza.

The term “adenovirus” as referred to herein indicates over 52 adenoviral subtypes isolated from humans, and as many from other mammals and birds. See, e.g., Strauss. “Adenovirus Infections in Humans,” in The Adenoviruses, Ginsberg, ed., Plenum Press, New York, N.Y., pp. 451-496 (1984). The term “adenovirus” can be referred to herein with the abbreviation “Ad” followed by a number indicating serotype, e.g., Ad5. As used herein, the term “oncolytic adenovirus” means an adenovirus that is an oncolytic virus, for example an adenovirus that can replicate or that it is replication-competent in a cancer cell. They are different from a non-replicating adenovirus because a non-replicating adenovirus is unable to replicate in the cancer cell. Non-replicating adenoviruses are used in gene therapy as carriers of genes to target cells, since the goal is to express the therapeutic gene within the intact cell and not the lysis of the cell. In contrast, the therapeutic action of oncolytic adenoviruses is based on the ability to replicate and to lyse the target cell, and thereby eliminate the cancer cell. As used herein, oncolytic adenoviruses include both replication-competent adenoviruses able to lyse cancer cells, even without selectivity, and oncolytic adenoviruses that replicate selectively in cancer cells. Non-limiting examples of an oncolytic adenoviruses include H101, a conditionally replicative adenovirus generated by both E1B and E3 gene deletion, which selectively infects and kills tumor cells through viral oncolysis (see, for example, U.S. Patent Application Publication No. US20040202663A1) and dl1520, a variant adenovirus where a fragment of 827 bp in E1b region is deleted so that it does not express E1b-55kDa protein (see, for example, US Patent Application Publication No.

US20030104625Al). The variant adenovirus dl1520 does not replicate in normal cells, but selectively replicate in cancer cells where the tumor-suppressor gene p53 is dysfunctional and eventually lyse cancer cells. See, more examples at U.S. Pat. No. US 11,000,560 B2.

Adeno-associated virus (AAV) is a non-pathogenic virus, so it is currently being investigated for many gene therapy applications including oncolytic cancer treatments due to its relatively safe nature. AAV can only package genomes between 2-5.2 kb in size when they are flanked with inverted terminal repeat sequences (ITRs), but optimally holds a genome of 4.1 to 4.9 kb in length. AAV has been researched as a viral vector in gene therapy for cancer treatment as an oncolytic virus, and is referred to herein as an oncolytic AAV. Examples of oncolytic AAV include AAV-2-TRAIL where the tumor necrosis factor-related apoptosis-inducing ligand (TRAIL) has been studied when delivered by AAV serotype 2 capsids on human cancer cell lines, AAV-2-HSV-TK where an AAV-2 vector expresses HSV-1 thymidine kinase (HSV-TK), a common anti-cancer therapy that converts the ganciclovir (GCV) into the toxic GCV-triphosphate within cells expressing the enzyme, AAV-2-sc39TK which is an AAV-2 vector expresses sc39TK, a five-codon substitution from HSV-TK where silent mutations have been introduced into the GCV-resistant spliced acceptor and donor sequences, and AAV-DC-CTL where an AAV is used to deliver CEA.

Reovirus is a nonenveloped segmented double stranded RNA virus from the Reoviridae family. As used herein, the term “oncolytic reovirus” refers to a reovirus capable of selectively replicating in dividing cells (e.g. a proliferative cell such as a cancer cell) with the aim of slowing the growth and/or lysing the dividing cell, either in vitro or in vivo, while showing no or minimal replication in non-dividing cells. Typically, an oncolytic reovirus contains a retroviral genome packaged into a viral particle (or virion) and is infectious (i.e. capable of infecting and entering into a host cell or subject). See, for example, US Patent Application Publication NO. US20210008136A1. One non-limiting example is the type 3 reovirus Dearing strain (T3D, Reolysin), which has been used in Phase I-III human studies for carcinomas and recurrent gliomas.

Measles or Measles virus (MV) is an enveloped negative sense RNA from the Paramyxoviridae family virus that is highly infectious. MV has been demonstrated safe in patient in several early phase clinical trials involving melanoma, ovarian carcinoma, and myeloma. MV is also in clinical trials in combination with cyclophosphamide for treatment of refectory multiple myeloma (NCT00450814) and in early phase studies for head and neck and breast cancer squamous cell carcinoma (NCT 01846091), malignant peripheral nerve sheath tumors (NCT02919449) as well as recurrent glioblastoma multiforme (NCT00390299).

The term “herpes simplex virus” or “HSV” as used herein means a herpes simplex virus that produces the effect of the present invention, which includes a wild type or mutant herpes simplex virus. In one embodiment, the mutant non-natural HSV is obtained by mutating or modifying any of the genes of wild-type HSV or by inserting any of exogenous genes. The serum type of HSV comprises, alternatively consists essentially of, or yet further consists of a type 1 HSV (or HSV-1) or a type 2 HSV (or HSV-2). The HSV-1 is an enveloped, double-stranded DNA virus. In one embodiment, the HSV-1 can infect a human cell. In another embodiment, a sequence, a gene or multiple genes can be incorporated to the HSV-1. The size of incorporated sequence can be approximate 1 base, 5 bases, 10 bases, 100 bases, 1 kb, 10 kb, 100 kb, or 150 kb. HSV-1 can induce cell lysis at a relatively low multiplicity of infection (MOI), and its proliferation can be inhibited by anti-viral drugs. In one embodiment, the HSV viral DNA stays outside the chromosomes without being incorporated into the genome of host cells. The HSV-1 can encompass a variety of strains (e.g., KOS and McKrae). See Wang et al., (2013) Virus Res. 173(2):436-440. In one embodiment, the HSV-1 is an HSV-1 KOS strain. In another embodiment, the HSV-1 is an HSV-1 McKrae strain.

There are several HSV mutants, for example, 17TermA HSV and rRp 450 HSV. The term “17TermA HSV” refers to mutant HSV-1 virus that comprises the entire ICP34.5 gene, but with a termination codon inserted before 100 bp of coding region, resulting in early termination of protein expression and expression of a 30 amino acid truncated protein. The 17TermA HSV mutant displays a growth defect because of the truncated ICP34.5 protein. See Orvedahl et al., (2007) Cell Host & Microbe, 1:1, 23-25. The term “rRp 450” refers to an attenuated herpes simplex 1 vector deficient in the viral-encoded ribonucleotide reductase or ICP6. See Aghi M et al., (1999) Cancer Res., 59(16):3861-5.

The HSV genome encodes multiple virulence proteins, which include but are not limited to glycoprotein E (“gE”), Infected Cell Protein 0 (“ICP0”), Infected Cell Protein 6 (“ICP6”), DNA packaging terminase subunit 1, Infected Cell Protein 8 (“ICPS”), and Infected Cell Protein 34.5 (“ICP 34.5”). An exemplary HSV 1 genome can be found at NCBI Reference Sequence: NC 001806.2, last accessed on Mar. 13, 2020.

In some embodiments, the HSV is an oncolytic HSV, such as C134 and G207, Other non-limiting examples of oncolytic HSV (oHSV) include HSV1716, R3616 (e.g. see Chou & Roizman, Proc. Natl. Acad. Sci. Vol. 89, pp. 3266-3270, April 1992), NV1020 (Geevarghese et al, Human Gene Therapy 2010 September; 21(9):1119-28), RE 6 (Thompson et al, Virology 131, 171-179 (1983)), Oncovex™ (Simpson et al, Cancer Res 2006; 66: (9) 4835-4842 May 1, 2006; Liu et al, Gene Therapy (2003): 10, 292-303), disptk, hrR3, R4009, MGH-1, MGH-2, G474, Myb34.5, DF3γ34.5, HF10, NV1042, RAMBO, rQNestin34.5, R5111, R-LM113, CEAICP4, CEAγ34.5, DF3γ34.5, KeM34.5 (Manservigi et al, The Open Virology Journal 2010; 4:123-156), rRp 450, M032 (Campadelli-Fiume et al, Rev Med. Virol 2011; 21:213-226), Bacol (Fu et al, Int. J. Cancer 2011; 129(6):1503-10) and M032. See, for example, U.S. Pat. No. 10,813,958B2.

The oncolytic HSV-1 C134 is a neurovirulent, oncolytic second-generation, replication-competent, recombinant and genetically-engineered herpes simplex virus type 1 (HSV-1) where the gene for ICP34.5 has been deleted and the gene encoding the human cytomegalovirus (HCMV), protein kinase R (PKR) evasion protein IRS1, with potential oncolytic and immunostimulating activities. Upon intratumoral administration, oncolytic HSV-1 C134 specifically infects and replicates within the rapidly dividing, glioma cells, thereby directly lysing tumor cells. The released virus particles, in turn, infect and replicate in neighboring tumor cells, thereby further killing tumor cells. Tumor antigens released from the lysed tumor cells also activate the immune system to induce a tumor-specific systemic immune and cytotoxic T-lymphocyte (CTL) response, thereby killing nearby non-infected tumor cells. Deletion of the gene encoding for ICP34.5 imparts tumor selectivity by preventing replication in healthy cells. IRS1 expression allows the virus to replicate within tumors but limits viral spread. See, for example, Cassady et al, The Open Virology Journal 2010; 4:103-108 and US Patent Application Publication No. US20080206199A1.

G207 is a neuroattenuated, replication-competent, recombinant herpes simplex virus-1(HSV-1) with potential oncolytic activity. Upon intracerebral administration, oncolytic HSV-1G207 preferentially replicates within glioma cells, which may elicit tumor-specific systemic immune and cytotoxic T lymphocyte (CTL) responses in addition to direct cytopathic effects. Derived from wild-type HSV-1 strain F, this agent has been neuroattenuated by deletions in both copies of the gamma34.5 gene, the major determinant of HSV neurovirulence. In addition, the HSV-1 gene UL39, encoding the viral ribonucleotide reductase large subunit infected cell protein 6 (ICP 6), has been inactivated through the insertion of the Escherichia coli lacZ gene. By inactivating UL39, viral ribonucleotide reductase activity is disrupted, resulting in the inhibition of nucleotide metabolism and viral DNA synthesis in nondividing cells but not in dividing cells. See, for example, Toda et al, Human Gene Therapy 9:2177-2185, Oct. 10, 1995.

“Newcastle Disease Virus” and “NDV” refer to a negative-sense single-stranded RNA virus of the family Paramyxoviridae that causes a highly contagious zoonotic bird disease affecting many domestic and wild avian species. Naturally-occurring NDV has been reported to be an effective oncolytic agent in a variety of animal tumor models (Sinkovics and Horvath, 2000, J Clin Virol 16: 1-15; Zamarin et al., 2009; Mol Ther 17: 697;

Elankumaran et al., 2010, J Virol 84: 3835; Schirrmacher et al., 2009, Methods Mol Biol 542:565; Bart et al., 1973, Nat New Biol 245: 229), Naturally-occurring strains of NDV bave been used in multiple clinical trials against advanced human cancers (Sinkovics and Horvath; Lorence et al. 2007. Curr Cancer Drug Targets 7: 157-167; Hotte et al. 2007 Clin Cancer Res 13: 977-985; Freeman et al. 2006. Mol Ther 13: 221-228; Pecora et al. 2002. J Clin Oncol 20: 2251-2266; Csatary et al. 2004. J Neurooncol 67:83-93). See, for example, U.S. Patent Application Publication No. US20200061184A1.

Vesiculovirus is a genus of negative-sense single-stranded RNA viruses in the family Rhabdoviridae, within the order Mononegavirales. A vesiculovirus comprises, or alternatively consists essentially of, or yet further consists of either or both of vesicular stomatitis virus or maraba virus.

Vesicular stomatitis virus (VSV) is a negative-stranded virus, comprising only 5 genes, that preferentially replicates in immortalized and malignant cells, eventually inducing apoptosis. The ability of VSV to reproduce in tumor or malignant cells has been reported to occur, in part, to a defective interferon (IFN) system, Since the IFN system is functional in normal cells, efficient replication of VSV, which is an IFN-sensitive virus, is prevented.

Based on in vitro and in vivo observations, it has been demonstrated that VSV effectively replicates in and lyses infected cancer cells, while leaving normal cells relatively unaffected. See, for example, Stodj et al., Nature Medicine 6:821-825, July 2000; Fernandez et al. 2002, J. Virol. 76:895-904; Balachandran et al. 2001, J. Virol, 75:3474-9; Balachandran and Barber, 2000, IUBMB Life 50: 135-8; and US Patent Application Publication No. 20160022748A1. Accordingly, VSV can serve as an oncolytic virus.

VSV and maraba virus are nonsegmented negative stranded virus in the Rhabdoviridae family that are being independently developed as oncolytic virus. In some embodiments, the maraba virus is maraba oncolytic virus expressing mutant HPV E6/E7, a cancer vaccine comprised of a recombinant, attenuated form of the oncolytic rhabdovirus Maraba (MG1) encoding inactive, mutant forms of the human papillomavirus (HPV) transforming proteins E6 and E7, with potential immunostimulating and antineoplastic activities. Upon administration of MG1-E6E7, MG1 preferentially infects tumor cells and induces the expression of the E6 and E7 proteins. The MG1 virus exerts its oncolytic activity, thereby directly lysing tumor cells. Following the lysis of infected cells, the virus is released and can infect adjacent cells, which both induces further tumor cell oncolysis and may activate the immune system to kill the infected tumor cells. The expressed E6 and E7 proteins stimulate the host immune system to mount a cytotoxic T-lymphocyte (CTL) response against tumor cells expressing HPV E6 and E7, thereby further inducing tumor cell lysis.

Oncoproteins E6 and E7 play a key role in the development of cervical intraepithelial neoplasia (CIN) and cervical carcinoma.

Poliovirus (PV), the causative agent of polio (also known as poliomyelitis), is a serotype of the species Enterovirus C, in the family of Picornaviridae. It comprises an RNA genome and a protein capsid. The genome is a single-stranded positive-sense RNA (+ssRNA) genome that is about 7500 nucleotides long. The viral particle is about 30 nm in diameter with icosahedral symmetry. Recombinant poliovirus that targets an intriguing aberration of translation control in malignancy has been generated and used as an oncolytic virus. See, for example, Goetz et al. Future Virol. 2011 September; 6(9):1045-1058.

ECHO-7 (RIGVIR) is a wild type member of the echovirus group of viruses. It was formerly approved as a virotherapy medication by the State Agency of Medicines of the Republic of Latvia (2004-2019). ECHO-7 virus is known to possess oncolytic activity in cutaneous melanoma. See for example, WO2003105875A1 and WO2015007788A1. It is a non-pathogenic virus Picornaviridae family, Enterovirus genus, Enteric Cytopathogenic Human Orphan group, type 7 (ECHO-7), group VII, positive-sense single-stranded RNA virus adapted to melanoma, with oncolytic, oncotropic and immunomodulating properties. See, for example, IDonina et al. Acta Medica Lituanica Suppl. 2002; 9:97-100;

Alberts et al. Eur J Pharmacol 2018; 837:117-126; Glinkina et al. Vopr Onkol. 1992; 38(5): 540-547; Glinkina et al. Vopr Onkol. 1992; 38(5):534-540; and Glinkina et al. Vopr Onkol. 1992; 38(6):659-666.

Coxsackievirus A21 is a preparation of naturally occurring, oncolytic enterovirus, with potential antineoplastic activity. Upon administration, coxsackievirus V937 targets and binds to intracellular adhesion molecule 1 (ICAM-1) and decay acceleration factor (DAF), both cell surface molecules that are overexpressed on certain malignant cells. After entering the cells, coxsackievirus V937 replicates in these cancer cells, thereby causing cancer cell lysis. This results in a reduction of tumor cell growth.

Senecavirus, is a genus of viruses in the order Picornavirales, in the family Picornaviridae. It is a replication-competent oncolytic picornavirus. It has selective tropism for cancers with neuroendocrine features including small cell lung cancer (SCLC) and several pediatric solid tumors including retinoblastoma, neuroblastoma, and medulloblastoma. See, for example, Melcher et al. Mol Ther. 2011 June; 19(6):1008-16. It also has potential antineoplastic activity. The initial isolate, SVV-001, is being developed as an anti-cancer therapeutic by Neotropix, Inc. under the name NTX-010 for cancers with neuroendocrine features including small cell lung cancer and a variety of pediatric solid tumors.

Pox virus or poxvirus is double stranded (ds) DNA virus with large well characterized genomes (130-300 kb in size) that produce both enveloped and non-enveloped forms of infectious virus. The Pox virus comprises, or alternatively consists essentially of, or yet further consists of one or more of: a vaccinia virus (VACV), a cowpox, a myxoma virus, or a racoonpox virus (RPV), squirrelpox virus, or yaba monkey tumor virus. Pox viruses rely on post-entry host response differences that restrict viral replication for conditional replication in tumor versus nonmalignant cells.

Retroviruses carry their genetic information in the form of RNA, however, once the virus infects a cell, the RNA is reverse-transcribed into the DNA form which integrates into the genomic DNA of the infected cell. The integrated DNA form is called a provirus. The retrovirus comprises gamma-retrovirus or lentivirus. Unlike other virotherapies, oncolytic retrovirus relies primarily on gene expression related activity by the virus solely, by delivering a gene or genes of interest selectively to replicating cells and integrating in the host cell genome. Replication competent retroviruses (RRVs) that target tumor-specific receptors for entry and then replicate locally within the tumor environment have been developed. The gene(s) expressed by the RR Vs can directly induce cancer cell damage and death. One example is Toca 511 (vocimagene amiretrorepvec), a gamma-retroviral replicating vector encoding cytosine deaminse.

Parvovirus is a nonenveloped single stranded (ss) DNA virus that includes both autonomous virus (e.g., parvovirus B19) and helper-dependent virus (e.g., AAV) within the Parvoviridae family. Autonomous parvoviruses exhibit both selective replication and direct oncolytic activity and an infection related immune-mediated response, thus serving as an oncolytic virus. One example is a rat parvovirus contract, ParvOryx, under investigation in treating progressive primary and recurrent glioblastoma multiforme (NCT01301430). Another example is parvovirus H-1, a replication-competent oncolytic parvovirus with potential antineoplastic activity. Upon infection of host cells, parvovirus H-1 preferentially replicates in tumor cells compared to healthy normal cells, thereby potentially resulting in tumor cell lysis and leading to an inhibition of tumor cell proliferation. In addition, HI-infected tumor cells strongly induce the release of the inducible heat shock protein 72 (Hsp72i), which chaperone tumor associated antigens in the HI-mediated tumor lysates and may activate antigen presenting cells (APCs), thereby leading to antitumor immune responses. Parvovirus H-1 does not cause any pathogenic effect in normal, healthy cells and is able to cross the blood brain barrier (BBB).

Influenza is a segmented single-stranded negative-sense RNA virus in the Orthomyxoviridae family. Influenza can rely on mutations in the cancer cells that disrupt molecular pathways involved in the antiviral response to permit selective replication in cancerous cells, thus serving as an oncolytic virus. In some embodiments, engineered mutation in the NS1 antiviral evasion gene attenuates the virus and permits this selective replication in cancerous cells with PKR and IFN-signaling defects or in the cells with RAS mutations or MAPK upregulations the suppress the PKR and IFN.

As used herein, the term “cytokine” refers to small proteins (about 5-20 kDa) important in cell signaling, including but not limited to chemokines, interferons, interleukins (ILs), lymphokines, and tumour necrosis factors, but generally not hormones. Cytokines are peptides and cannot cross the lipid bilayer of cells to enter the cytoplasm.

As used herein, an anti-cancer cytokine refers to a cytokine suitable for use in treating a cancer optionally in combination with another cancer therapy. In some embodiments, the anti-cancer cytokine comprises, or alternatively consists essentially of, or yet further consists of any one or more of: interleukin 2 (IL-2), Interleukin 5 (IL-5), interleukin 12 (IL-12), interleukin 15 (IL-15), Interleukin 18 (IL-18), interleukin 21 (IL-21), interleukin 27 (IL-27), Interferon α (IFNα), Type I interferon (IFNI), Type II interferon (IFNII), Interferon γ (IFNγ), Interferon β (IFNβ), CD40 Ligand (CD40L), Colony Stimulating Factor 2 (CSF2), RANTES, Tumor Necrosis Factor α (TNFα), C-X-C Motif Chemokine Ligand 9 (CXCL9), C-X-C Motif Chemokine Ligand 10 (CXCL10), or C-X-C Motif Chemokine Ligand 11 (CXCL11).

Interleukin-2 (IL-2) is an interleukin, a type of cytokine signaling molecule in the immune system. It is a 15.5-16 kDa protein that regulates the activities of white blood cells (leukocytes, often lymphocytes) that are responsible for immunity. In some embodiments, the IL-2 is a human IL-2. In some embodiments, the IL-2 is of other species, such as a chimpanzee IL-2 having an NCBI Reference Sequence of XP_517425.1. Non-limiting exemplary sequences of this protein or the underlying gene can be found under Gene Cards ID: GC04M122451, HGNC (6001), NCBI Entrez Gene (3558), Ensembl (ENSG00000109471), OMIM® (147680), UniProtKB/Swiss-Prot (P60568), and Open Targets Platform (ENSG00000109471), each of which is incorporated by reference herein in its entirety.

Interleukin 5 (IL-5) is an interleukin (IL) produced by type-2 T helper cells and mast cells. It acts as a growth and differentiation factor for both B cells and cosinophils; plays a major role in the regulation of eosinophil formation, maturation, recruitment and survival; and functions by binding to its receptor, which is a heterodimer, whose beta subunit is shared with the receptors for interleukin 3 (IL3) and colony stimulating factor 2 (CSF2/GM-CSF). The increased production of this cytokine may be related to pathogenesis of eosinophil-dependent inflammatory diseases. In some embodiments, the IL-S is a human IL-5. Non-limiting exemplary sequences of this protein or the underlying gene can be found under Gene Cards ID: C05M132541, HGNC: 6016, NCBI Entrez Gene: 3567, Ensembl: ENSG 00000113525, OMIM®: 147850, or UniProtKB/Swiss-Prot: P05113, each of which is incorporated by reference herein in its entirety.

Interleukin 12 (IL-12) is an interleukin that is naturally produced by dendritic cells, macrophages, neutrophils, and human B-lymphoblastoid cells (NC-37) in response to antigenic stimulation. In some embodiments, the IL-12 is a human IL-12. It is a heterodimeric cytokine comprising, or alternatively consisting essentially of, or yet further consisting of IL-12A (p35) and IL-12B (p40). Non-limiting exemplary sequences of IL12A or the underlying gene can be found under Gene Cards ID: GC03P159988, GNC: 5969, NCBI Entrez Gene: 3592, Ensembl: ENSG00000168811, OMIM®: 161560, or UniProtKB/Swiss-Prot: P29459, each of which is incorporated by reference herein in its entirety. Non-limiting exemplary sequences of IL 12B or the underlying gene can be found under Gene Cards ID: GC05M159314, HGNC: 5970, NCBI Entrez Gene: 3593, Ensembl: ENSG00000113302, OMIM®: 161561, or UniProtKB/Swiss-Prot: P29460, each of which is incorporated by reference herein in its entirety.

Interleukin-15 (IL-15) is a cytokine with structural similarity to Interleukin-2 (IL-2). Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following infection by virus(es). This cytokine induces the proliferation of natural killer cells. In some embodiments, the IL-15 is a human IL-15. Non-limiting exemplary sequences of this protein or the underlying gene can be found under Gene Cards ID: GC04P141636, HGNC: 5977, NCBI Entrez Gene: 3600, Ensembl: ENSG00000164136, OMIM®: 600554, or UniProtKB/Swiss-Prot: P40933, each of which is incorporated by reference herein in its entirety.

Interleukin 18 (IL-18) is a proinflammatory cytokine. Many cell types, both hematopoietic cells and non-hematopoietic cells, have the potential to produce IL-18. IL-18 can modulate both innate and adaptive immunity and its dysregulation can cause autoimmune or inflammatory diseases. In some embodiments, the IL-18 is a human IL-18.Non-limiting exemplary sequences of this protein or the underlying gene can be found under Gene Cards ID: GC11M112143, HGNC: 5986, NCBI Entrez Gene: 3606, Ensembl: ENSG00000150782, OMIM®: 600953, UniProtKB/Swiss-Prot: Q14116, each of which is incorporated by reference herein in its entirety.

Interleukin-21 (IL-21) is a cytokine that has potent regulatory effects on cells of the immune system, including natural killer (NK) cells and cytotoxic T cells that can destroy virally infected or cancerous cells. This cytokine induces cell division/proliferation in its target cells. IL-21 signaling and biological effects on target cells include, but are not limited to, B cell proliferation, immunoglobulin production, T follicular helper (Tfh) cell proliferation, cytotoxic T cell (CTL) proliferation and anti-tumor activity, NK proliferation and antibody-dependent cellular cytotoxicity (ADCC) activity, and suppression of regulatory T cells (Tregs) proliferation. More details can be found in Croce M, et al. J Immunol Res. 2015; 2015:696578. For example, IL-21 released by CD4 T cells binds to a complex formed by IL-21R, γc, JAK-1 and JAK-3. By phosphorylating STAT-5, STAT-3 or STAT-1, various IL-21 target genes are expressed, such as GzmA, GzmB, IL-10, Bim, Socs, Bx1-6, Jak3 etc. Accordingly, certain immune enhancing effects are achieved, including, but not limited to, for B cells: cell proliferation, immunoglobulin production, plasma cell differentiation; for Tfh cells, cell differentiation and proliferation; for CTL, cell proliferation, survival, anti-tumor activity, CD28 and L-selectin expression, for NK cells: proliferation, antitumor activity, ADCC activity; for Th17 cells: differentiation, proliferation IL-23R expression; and for Treg: inhibiting survival, inhibiting generation. Additionally, certain immune regulatory effects are also achieved, including, but not limited to, for T regulatory type 1 (Tr1) cells: differentiation, proliferation, IL-10 production; for dendritic cells (DCs): apoptosis, inhibition of antigen-presenting cell (APC) APC function; and for regulatory B cells (Breg/B10): differentiation, proliferation, and IL-10 production. In some embodiments, the IL-21 is a human IL-21. Non-limiting exemplary sequences of this protein or the underlying gene can be found under Gene Cards ID: GC04M122612, HGNC: 6005, NCBI Entrez Gene: 59067, Ensembl: ENSG00000138684, OMIM®: 605384, and UniProtKB/Swiss-Prot: Q9HBE4,each of which is incorporated by reference herein in its entirety.

Interleukin 27 (IL-27) is a member of the IL-12 cytokine family. It is a heterodimeric cytokine that is composed of two distinct genes, Epstein-Barr virus-induced gene 3 (EBI3) and IL-27p28. IL-27 is expressed by antigen presenting cells and interacts with a specific cell-surface receptor complex known as IL-27 receptor (IL-27R). IL-27 induces differentiation of the diverse populations of T cells in the immune system and also upregulates IL-10. In some embodiments, the IL-27 is a human IL-27. Non-limiting exemplary sequences of this protein or the underlying gene can be found under Gene Cards ID: GC16M028514, HGNC: 19157, NCBI Entrez Gene: 246778, Ensembl: ENSG00000197272, OMIM®: 608273, or UniProtKB/Swiss-Prot: Q8NEV9, each of which is incorporated by reference herein in its entirety.

Interferon α (IFNα) is produced mainly by plasmacytoid dendritic cells (pDCs). They are mainly involved in innate immunity against viral infection. The genes responsible for their synthesis come in 13 subtypes that are called IFNA1 (Genecards ID (GCID): GC09P021522, UniProtKB/Swiss-Prot: P01562), IFNA2 (GCID: GC09M021384, niProtKB/Swiss-Prot: P01563), IFNA4 (GCID: GC09M021186, UniProtKB/Swiss-Prot: P05014), IFNA5 (GCID: GC09M021304, UniProtKB/Swiss-Prot: P01569), IFNA6 (GCID: GC09M021349, UniProtKB/Swiss-Prot: P05013), IFNA7 (GCID: GC09M021473, UniProtKB/Swiss-Prot: P01567), IFNA8 (GCID: GC09P021409, UniProtKB/Swiss-Prot: P32881), IFNA10 (GCID: GC09M021474, UniProtKB/Swiss-Prot: P01566), IFNA13 (GCID: GC09M021367, UniProtKB/Swiss-Prot: P01562), IFNA14 (GCID: GC09M021239, UniProtKB/Swiss-Prot: P01570), IFNA16 (GCID: GC09M021216, UniProtKB/Swiss-Prot: P05015), IFNA17 (GCID: GC09M021227, UniProtKB/Swiss-Prot: P01571), and IFNA21 (GCID: GC09M021165, UniProtKB/Swiss-Prot: P01568). These genes are found together in a cluster on chromosome 9. In some embodiments, the IFNa is a human IFNo.

Each of the Genecard and UniProt webpages following the identified reference numbers is incorporated by references herein in its entirety.

The term “type I interferon” or “IFNI”′ as used herein is intended to refer to members of the type I interferon family of molecules that that binds to a cell surface receptor complex known as the IFN-α receptor (IFNAR) that consists of IFNARI and IFNAR2 chains. Non-limiting examples of type I interferon include IFN-α (such as IFN-α1 (Genecards ID (GCID): GC09P021522, UniProtKB/Swiss-Prot: P01562), IFN-α2 (GCID: GC09M021384, niProtKB/Swiss-Prot: P01563), IFN-α4 (GCID: GC09M021186, UniProtKB/Swiss-Prot: P05014), IFN-α5 (GCID: GC09M021304, UniProtKB/Swiss-Prot: P01569), IFN-α6 (GCID: GC09M021349, UniProtKB/Swiss-Prot: P05013), IFN-α7 (GCID: GC09M021473, UniProtKB/Swiss-Prot: P01567), IFN-α8 (GCID: GC09P021409, UniProtKB/Swiss-Prot: P32881), IFN-α10 (GCID: GC09M021474, UniProtKB/Swiss-Prot: P01566), IFN-α13 (GCID: GC09M021367, UniProtKB/Swiss-Prot: P01562), IFN-α14 (GCID: GC09M021239, UniProtKB/Swiss-Prot: P01570), IFN-α16 (GCID: GC09M021216, UniProtKB/Swiss-Prot: P05015), IFN-α17 (GCID: GC09M021227, UniProtKB/Swiss-Prot: P01571), and IFN-α21 (GCID: GC09M021165, UniProtKB/Swiss-Prot: P01568)), IFN-β (such as IFN-β1 (GCID: GC09M021077, UniProtKB/Swiss-Prot: P01574) or IFN-β3 (UniProtKB/Swiss-Prot: T2K1E0)), IFN-κ (GCID: GC09P027514, UniProtKB/Swiss-Prot: Q9POWO) , IFN-δ, IFN-ε (GCID: GC09M021480, UniProtKB/Swiss-Prot: Q86WN2), IFN-τ, IFN-ω (GCID: GC09M021140, UniProtKB/Swiss-Prot: P05000), and IFN-ζ. In some embodiments, the type I interferon is a human type I interferon. Each of the Genecard and UniProt webpages following the identified reference numbers is incorporated by references herein in its entirety.

The term “type II interferon” denotes interferons that bind to the interferon-gamma receptor (IFNGR). The type II interferons present in humans comprise interferon γ. Interferon γ (IFNγ) is a soluble cytokine that is a member of the type II interferon class. It is secreted by cells of both the innate and adaptive immune systems. The active protein is a homodimer that binds to the interferon gamma receptor which triggers a cellular response to viral and microbial infections. In some embodiments, the IFNγ is a human IFNγ. Non-limiting exemplary sequences of this protein or the underlying gene can be found under Gene Cards ID: GC12M068154, HGNC: 5438, NCBI Entrez Gene: 3458, Ensembl: ENSG00000111537, OMIM®: 147570, or UniProtKB/Swiss-Prot: P01579, each of which is incorporated by reference herein in its entirety.

Interferon β (IFNβ) is a cytokine that belongs to the interferon family of signaling proteins, which are released as part of the innate immune response to pathogens, and has antiviral, antibacterial, and anticancer properties. In some embodiments, the IFNβ is a human IFNβ. Non-limiting exemplary sequences of this protein or the underlying gene can be found under Gene Cards ID: GC09M021077, HGNC: 5434, NCBI Entrez Gene: 3456, Ensembl: ENSG00000171855, OMIM®: 147640, or UniProtKB/Swiss-Prot: P01574, each of which is incorporated by reference herein in its entirety.

CD40 Ligand (CD40L) is a protein that is primarily expressed on activated T cells and is a member of the TNF superfamily of molecules. It binds to CD40 (protein) on antigen-presenting cells (APC), which leads to many effects depending on the target cell type. In total CD40L has three binding partners: CD40, α5β1 integrin and αaIIbβ3. CD154 acts as a costimulatory molecule and is particularly important on a subset of T cells called T follicular helper cells (T_{FH} cells). On T_{FH} cells, CD154 promotes B cell maturation and function by engaging CD40 on the B cell surface and therefore facilitating cell-cell communication. In some embodiments, the CD40L is a human CD40L. Non-limiting exemplary sequences of this protein or the underlying gene can be found under Gene Cards ID: GC0XP136649, HGNC: 11935, NCBI Entrez Gene: 959, Ensembl: ENSG00000102245, OMIM®: 300386, or UniProtKB/Swiss-Prot: P29965, each of which is incorporated by reference herein in its entirety.

Colony Stimulating Factor 2 (CSF2) is a monomeric glycoprotein secreted by macrophages, T cells, mast cells, natural killer cells, endothelial cells and fibroblasts that functions as a cytokine stimulating stem cells to produce granulocytes (neutrophils, eosinophils, and basophils) and monocytes. In some embodiments, the CSF2 is a human CSF2. Non-limiting exemplary sequences of this protein or the underlying gene can be found under Gene Cards ID: GC05P132073, HGNC: 2434,NCBI Entrez Gene: 1437, Ensembl: ENSG00000164400, OMIM®: 138960, or UniProtKB/Swiss-Prot: P04141, each of which is incorporated by reference herein in its entirety.

RANTES is also referred to as C-C Motif Chemokine Ligand 5 or CCL5. It is an 8 kDa protein classified as a chemotactic cytokine or chemokine. CCL5 is chemotactic for T cells, eosinophils, and basophils, and plays an active role in recruiting leukocytes into inflammatory sites. With the help of particular cytokines (i.e., IL-2 and IFN-γ) that are released by T cells, CCL5 also induces the proliferation and activation of certain natural-killer (NK) cells to form CHAK (CC-Chemokine-activated killer) cells. It is also an HIV-suppressive factor released from CD8+ T cells. In some embodiments, the RANTES is a human RANTES. Non-limiting exemplary sequences of this protein or the underlying gene can be found under Gene Cards ID: GC17M035871, GNC: 10632, NCBI Entrez Gene: 6352, Ensembl: ENSG00000271503, OMIM®: 187011, or UniProtKB/Swiss-Prot: P13501, each of which is incorporated by reference herein in its entirety.

Tumor Necrosis Factor α (TNFα, or TNF) is a cytokine released by macrophages upon detection of an infection to alert other immune system cells as part of an inflammatory response. TNF is a member of the TNF superfamily, which consists of various transmembrane proteins with a homologous TNF domain. In some embodiments, the TNFα is a human TNFα. Non-limiting exemplary sequences of this protein or the underlying gene can be found under Gene Cards ID: GC06P055202, HGNC: 11892, NCBI Entrez Gene: 7124, Ensembl: ENSG00000232810, OMIM®: 191160, or UniProtKB/Swiss-Prot: P01375, each of which is incorporated by reference herein in its entirety.

C-X-C Motif Chemokine Ligand 9 (CXCL9) is a small cytokine belonging to the CXC chemokine family that is also known as monokine induced by gamma interferon (MIG). The CXCL9 is one of the chemokine which plays role to induce chemotaxis, promote differentiation and multiplication of leukocytes, and cause tissue extravasation. The CXCL9/CXCR3 receptor regulates immune cell migration, differentiation, and activation. Immune reactivity occurs through recruitment of immune cells, such as cytotoxic lymphocytes (CTLs), natural killer (NK) cells, NKT cells, and macrophages. In some embodiments, the CXCL9 is a human CXCL9. Non-limiting exemplary sequences of this protein or the underlying gene can be found under Gene Cards ID: GC04M076001, HGNC: 7098, NCBI Entrez Gene: 4283, Ensembl: ENSG00000138755, OMIM®: 601704, or UniProtKB/Swiss-Prot: Q07325, each of which is incorporated by reference herein in its entirety.

C-X-C Motif Chemokine Ligand 10 (CXCL10) involves in NK cell/T cell recruitment. It directs the activity of the effective CD8 and CD4 T cells and is an antiangiogenic/anti-tumor protein. In some embodiments, at the tumor site or inflammation site, CSCL10 is released, binds to CXCR3 on CD8 positive T cells resulting in release of granzyme B and IFNγ, and binds to CXCR3 on CD4 positive T cells resulting in release of IFNγ. See, for example, Bagheri et al. Cell Oncol (Dordr). 2020 June; 43(3):353-365. In some embodiments, the CXCL10 is a human CXCL10. Non-limiting exemplary sequences of this protein or the underlying gene can be found under Gene Cards ID: GC04M076021, HGNC: 10637, NCBI Entrez Gene: 3627, Ensembl: ENSG00000169245, OMIM®: 147310, or UniProtKB/Swiss-Prot: P02778, each of which is incorporated by reference herein in its entirety.

C-X-C Motif Chemokine Ligand 11 (CXCL11) is a small cytokine belonging to the CXC chemokine family that is also called Interferon-inducible T-cell alpha chemoattractant (I-TAC) and Interferon-gamma-inducible protein 9 (IP-9). It is highly expressed in peripheral blood leukocytes, pancreas and liver, with moderate levels in thymus, spleen and lung and low expression levels were in small intestine, placenta and prostate. In some embodiments, the CXCL11 is a human CXCL11. Non-limiting exemplary sequences of this protein or the underlying gene can be found under Gene Cards ID: GCID: GC04M076033, HGNC: 10638, NCBI Entrez Gene: 6373, Ensembl: ENSG00000169248, OMIM®: 604852, or UniProtKB/Swiss-Prot: 014625, each of which is incorporated by reference herein in its entirety

As used herein, the term “transcriptional regulator” refers to a molecule, such as a protein, that control gene expression. In some embodiments, a transcriptional regulator can activate a gene, referring to the transcriptional regulator activating or positively regulating expression of the gene. In some embodiments, a transcriptional regulator can decrease a gene, referring to the transcriptional regulator repressing or negatively regulating expression of the gene. Accordingly, an anti-cancer transcriptional regulator refers to a transcriptional regulator capable of treating a cancer alone or in combination with another cancer therapy.

In some embodiments, the anti-cancer transcriptional regulator activates one or more of: an anti-cancer cytokine, Interferon Regulatory Factor 7 (IRF7, Genecard (GC) ID (GCID): GC11M000612, UniProtKB/Swiss-Prot: Q92985), Interferon Alpha And Beta Receptor Subunit 1 (IFNAR1, GCID: GC21P033324, UniProtKB/Swiss-Prot: P17181), Toll-like receptor 3 (TLR 3, GCID: GC04P186059, UniProtKB/Swiss-Prot: 015455), Interleukin 7 Receptor (IL7R, GCID: GC05P035852, UniProtKB/Swiss-Prot: P16871), CD40 (GCID: GC20P046118, UniProtKB/Swiss-Prot: P25942), Interferon regulatory factor 3 (IFR3, GCID: GC19M049659, UniProtKB/Swiss-Prot: Q14653), Free Fatty Acid Receptor 3 (FFAR 3, GCID: GC19P 040640, UniProtKB/Swiss-Prot: 014843), CD28 (GCID: GC02P203706, UniProtKB/Swiss-Prot: P10747), CD3 (see, for example, GCID: GC11P118344, UniProtKB/Swiss-Prot: P09693 for CD3γ, GCID: GC11M118338, UniProtKB/Swiss-Prot: P04234 for CD38; and GCID: GC11P118304, UniProtKB/Swiss-Prot: P07766 for CD38), Interleukin 1 (IL-18, GCID: GC02M112829, UniProtKB/Swiss-Prot: P01584), PAF1 Homolog, Paf1/RNA Polymerase II Complex Component (PAF1, GCID: GC19M039385, UniProtKB/Swiss-Prot: Q8N7H5), Prostaglandin E Receptor 2 (PTGER2, GCID: GC14P052314, UniProtKB/Swiss-Prot: P43116), CD5 (GCID: GC11P061114, niProtKB/Swiss-Prot: P06127) or T cell Receptor (TCR, see for example, GCID: GC14P021621, UniProtKB/Swiss-Prot: PODTU3 for T-Cell Antigen Receptor, Alpha Polypeptide; GCID: GC07P146007, UniProtKB/Swiss-Prot: PODTU4 for T-Cell Antigen Receptor, Beta Polypeptide; GCID: GC14P022437 for T-Cell Antigen Receptor, Delta Polypeptide; and GCID: GC07M038240 for T-Cell Antigen Receptor, Gamma Polypeptide). Each of the Genecard and UniProt webpages following the identified reference numbers is incorporated by references herein in its entirety.

In some embodiments, the anti-cancer transcriptional regulator decreases one or more of: Prostaglandin E Receptor 4 (PTGER4, Genecard (GC) ID (GCID): GCID: GC05P040679, UniProtKB/Swiss-Prot: P35408), Polyribonucleotide Nucleotidyltransferase 1 (PNPT1, GCID: GC02M055634, UniProtKB/Swiss-Prot: Q8TCS8), Interleukin 10 Receptor Subunit α (IL10Rα, GCID: GC11P117987, UniProtKB/Swiss-Prot: Q13651), or Insulin Induced Gene 1 (INSIG1, GCID: GC07P155297, UniProtKB/Swiss-Prot: 015503). Each of the Genecard and UniProt webpages following the identified reference numbers is incorporated by references herein in its entirety.

As used herein, “Immune cells” includes, e.g., white blood cells (leukocytes, such as granulocytes (neutrophils, eosinophils, and basophils), monocytes, lymphocytes (T cells, B cells, natural killer (NK) cells and NKT cells)), and myeloid-derived cells (neutrophil, eosinophil, basophil, monocyte, macrophage, and dendritic cells). In some embodiments, the immune cell comprises, or alternatively consists essentially of, or yet further consists of one or more of: an NK cell, an NKT cell, a T cell, a B cell, a dendritic cell, a splenocyte or a macrophage.

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells may either be isolated or obtained from a commercially available source. “T cell” includes all types of immune cells expressing CD3 including T-helper cells (CD4+cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. T cells can either be isolated or obtained from a commercially available source.

As used herein, the term “splenocyte” refers to different white blood cell types in the spleen or purified from splenic tissue.

As used herein, a “cell surface marker” refers to a molecule (e.g., a protein) that is in contact with a cell and at least partially at the surface of the cell, and can be detected to distinguish the cell from one or more other types of cell from the same individual. In some embodiments, the presence and/or absence of a cell surface marker can be used to distinguish the cell from other types of cells. In some embodiments, the quantity of cell surface marker present on the surface of a cell can be used to distinguish the cell from other types of cells. In some embodiments, the cell surface marker is a transmembrane protein or a lipid-anchored protein. In some embodiments, the cell surface marker can be used to distinguish an immune cell from a non-immune cell.

Cluster of Differentiation (CD) 3 (CD3) is a protein complex and T cell co-receptor that is involved in activating both the cytotoxic T cell (CD8+ naive T cells) and T helper cells (CD4+ naive T cells) and composed of four distinct chains. It has been used as a negative marker for NK cells. Suitable antibodies for detecting such protein is available to one of skill in the art, such as from abcam (e.g., ab135372), BIOLEGEND® (e.g., UCHT1), or Thermo Fisher Scientific (e.g., 14-0037-82).

CD28 (Cluster of Differentiation 28) is one of the proteins expressed on T cells that provide co-stimulatory signals required for T cell activation and survival. T cell stimulation through CD28 in addition to the T-cell receptor (TCR) can provide a potent signal for the production of various interleukins (IL-6 in particular). CD28 is also the receptor for CD80 (B7.1) and CD86 (B7.2) proteins. When activated by Toll-like receptor ligands, the CD80 expression is upregulated in antigen-presenting cells (APCs). In some embodiments, the CD28 is a human CD28. Non-limiting exemplary sequences of this protein or the underlying gene can be found under Gene Cards ID: GC02P203706, HGNC: 1653, NCBI Entrez Gene: 940, Ensembl: ENSG00000178562, OMIM®: 186760, or UniProtKB/Swiss-Prot: P10747, each of which is incorporated by reference herein in its entirety.

The terms “4-1BBL,” “Tumor Necrosis Factor Superfamily Member 9,” “TNFSF9,” or “4-1BBL polypeptide” is a type 2 transmembrane glycoprotein receptor that is found on APCs (antigen presenting cells) and binds to 4-1BB (also known as CD137). The 4-1BB/4-1BBL complex belongs to the TNFR: TNF superfamily, which is expressed on activated T Lymphocytes. In some embodiments, the 4-1BBL is a human 4-1BBL. Non-limiting exemplary sequences of this protein or the underlying gene can be found under Gene Cards ID: GC19P006531, HGNC: 11939, NCBI Entrez Gene: 8744, Ensembl: ENSG00000125657, OMIM®: 606182, or UniProtKB/Swiss-Prot: P41273, each of which is incorporated by reference herein in its entirety.

CD16, also known as FcγRIII, is a cluster of differentiation molecule found on the surface of natural killer cells, neutrophils, monocytes, and macrophages and a molecule of the immunoglobulin superfamily (IgSF) involved in antibody-dependent cellular cytotoxicity (ADCC). CD16 has been identified as Fc receptors FcγRIIIa (CD16a) and FcγRIIIb (CD16b), which participate in signal transduction. In some embodiments, the Cd16 is a human CD16. Non-limiting exemplary sequences of CD16a or the underlying gene or suitable antibodies for detection of the protein can be found under Gene Cards ID: GC01M161541, HGNC: 3619, NCBI Entrez Gene: 2214, Ensembl: ENSG00000203747, OMIM®: 146740, or UniProtKB/Swiss-Prot: P08637, each of which is incorporated by reference herein in its entirety. Non-limiting exemplary sequences of CD16b or the underlying gene can be found under Gene Cards ID: GC01M161623, HGNC: 3620, NCBI Entrez Gene: 2215, Ensembl: ENSG00000162747, OMIM®: 610665, or UniProtKB/Swiss-Prot: O75015, each of which is incorporated by reference herein in its entirety.

NKG2D is an activating receptor on the NK cell surface. In some embodiments, the NKG2D is a human NKG2D. Non-limiting exemplary sequences of this protein or the underlying gene or suitable antibodies for detection of the protein can be found under Gene Cards ID: GC12M015056, HGNC: 18788, NCBI Entrez Gene: 22914, Ensembl: ENSG00000213809, OMIM®: 611817, or UniProtKB/Swiss-Prot: P26718, each of which is incorporated by reference herein in its entirety.

CD64 (Cluster of Differentiation 64) is a type of integral membrane glycoprotein known as an Fc receptor that binds monomeric IgG-type antibodies with high affinity. It is more commonly known as Fc-gamma receptor 1 (FcγRI). After binding IgG, CD64 interacts with an accessory chain known as the common γ chain (γchain), which possesses an ITAM motif that is necessary for triggering cellular activation. In some embodiments, the CD64 is a human CD64. Non-limiting exemplary sequences of this protein or the underlying gene or suitable antibodies for detection of the protein can be found under Gene Cards ID: GC01P149754, HGNC: 3613, NCBI Entrez Gene: 2209, Ensembl: ENSG00000150337, OMIM®: 146760, or UniProtKB/Swiss-Prot: P12314, each of which is incorporated by reference herein in its entirety.

“An immune cell engager” refers to one or more binding specificities that bind and/or activate an immune cell, e.g., a cell involved in an immune response. In embodiments, the immune cell is chosen from a T cell, an NK cell, a B cell, a dendritic cell, and/or the macrophage cell. The immune cell engager can be an antibody molecule, a receptor molecule (e.g., a full length receptor, receptor fragment, or fusion thereof (e.g., a receptor-Fc fusion)), or a ligand molecule (e.g., a full length ligand, ligand fragment, or fusion thereof (e.g., a ligand-Fc fusion)) that binds to the immune cell antigen (e.g., the NK cell antigen, the B cell antigen, the dendritic cell antigen, and/or the macrophage cell antigen). In embodiments, the immune cell engager specifically binds to the target immune cell, e.g., binds preferentially to the target immune cell. For example, when the immune cell engager is an antibody molecule, it binds to the immune cell antigen (e.g., the NK cell antigen, the B cell antigen, the dendritic cell antigen, and/or the macrophage cell antigen) with a dissociation constant of less than about 10 nM, and more typically, 10-100 pM.

As used herein, the term “specifically binding,” refers to the interaction between binding pairs (e.g., an antibody and an antigen, or a receptor and a ligand). In various instances, specifically binding can be embodied by an affinity constant of about 10⁻⁶ moles/liter, about 10⁻⁷ moles/liter, or about 10⁻⁸ moles/liter, or less.

The terms “equivalent” or “biological equivalent” are used interchangeably when referring to a particular molecule, biological, or cellular material and intend those having minimal homology while still maintaining desired structure or functionality. Non-limiting examples of equivalent polypeptides, include a polypeptide having at least 60%, or alternatively at least 65%, or alternatively at least 70%, or alternatively at least 75%, or alternatively 80%, or alternatively at least 85%, or alternatively at least 90%, or alternatively at least 95% identity thereto or for polypeptide sequences, or a polypeptide which is encoded by a polynucleotide or its complement that hybridizes under conditions of high stringency to a polynucleotide encoding such polypeptide sequences. Conditions of high stringency are described herein and incorporated herein by reference. Alternatively, an equivalent thereof is a polypeptide encoded by a polynucleotide or a complement thereto, having at least 70%, or alternatively at least 75%, or alternatively 80%, or alternatively at least 85%, or alternatively at least 90%, or alternatively at least 95% identity, or at least 97% sequence identity to the reference polynucleotide, e.g., the wild-type polynucleotide.

Non-limiting examples of equivalent polynucleotides having at least 60%, or alternatively at least 65%, or alternatively at least 70%, or alternatively at least 75%, or alternatively 80%, or alternatively at least 85%, or alternatively at least 90%, or alternatively at least 95%, or alternatively at least 97%, identity to a reference polynucleotide. An equivalent also intends a polynucleotide or its complement that hybridizes under conditions of high stringency to a reference polynucleotide.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. In certain embodiments, default parameters are used for alignment. A non-limiting exemplary alignment program is BLAST, using default parameters. In particular, exemplary programs include BLASTN and BLASTP, using the following default parameters: Genetic code=standard; filter-none; strand=both; cutoff=60, expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Sequence identity and percent identity can determined by incorporating them into clustalW (available at the web address:genome.jp/tools/clustalw/, last accessed on Jan. 13, 2017).

“Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure. In some embodiments, BLAST (accessible at blast.ncbi.nlm.nih.gov/Blast.cgi) or Clustal Omega (accessible at www.ebi.ac.uk/Tools/msa/clustalo/) are used in determining the identity. In further embodiments, default setting is applied.

“Homology” or “identity” or “similarity” can also refer to two nucleic acid molecules that hybridize under stringent conditions.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25 °C to about 37 °C; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40 °C to about 50 °C; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55 °C to about 68 °C; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” or “intracellular signaling domain” means any oligopeptide or polypeptide known to function as a domain that transmits a signal to cause activation or inhibition of a biological process in a cell. In certain embodiments, the intracellular domain may comprise, alternatively consist essentially of, or yet further comprise one or more costimulatory signaling domains in addition to the primary signaling domain. In some embodiments, CAR T cell refers to a T cell expressing a CAR.

As used herein, the term “cancer antigen” refers to an antigen expressed by a cancer cell. In further embodiments, the cancer antigen is not expressed in a non-cancer cell, or expressed at a lower level in a non-cancer cell compared to a cancer cell.

In some embodiments, the cancer antigen comprises, or alternatively consists essentially of, or yet further consists of one or more of: an embryonic antigen, an epithelial tumor antigen (ETA), melanoma-associated antigen (MAGE), or an abnormal product of ras. Additionally or alternatively, the cancer antigen is a human cancer antigen. In some embodiments, the cancer antigen comprises, or alternatively consists essentially of, or yet further consists of one or more of: alphafetoprotein (AFP, Genecards ID (GCID): GC04P073431, UniProtKB/Swiss-Prot: P02771), CA-125 (Mucin 16, Cell Surface Associated, Genecards ID (GCID): GC19M008848, UniProtKB/Swiss-Prot: Q8WX17), MUC-1 (Mucin 1, Cell Surface Associated, Genecards ID (GCID): GC01M155185,UniProtKB/Swiss-Prot: P15941), Tyrosinase (Genecards ID (GCID): GC11P089177,UniProtKB/Swiss-Prot: P14679), p53 (Tumor Protein P53, Genecards ID (GCID): GC17M007661, UniProtKB/Swiss-Prot: P04637), CD10 (Genecards ID (GCID): GC03P155024, UniProtKB/Swiss-Prot: P08473), CD19 (Genecards ID (GCID): GC16P032270, UniProtKB/Swiss-Prot: P15391), CD20 (Genecards ID (GCID): GC11P060475, UniProtKB/Swiss-Prot: P11836), CD21 (Genecards ID (GCID): GC01P207454, UniProtKB/Swiss-Prot: P20023), CD22 (Genecards ID (GCID): GC19P035319, UniProtKB/Swiss-Prot: P20273), CD25 (Genecards ID (GCID): GC10M006010, UniProtKB/Swiss-Prot: P01589), CD30 (Genecards ID (GCID): GC01P012063, UniProtKB/Swiss-Prot: P28908), CD33 (Genecards ID (GCID): GC19P051212, UniProtKB/Swiss-Prot: 20138), CD34 (Genecards ID (GCID); GC01M207880, UniProtKB/Swiss-Prot: P28906), CD37 (Genecards ID (GCID): GC19P049335, UniProtKB/Swiss-Prot: P11049), CD44v6 (an isoform containing the variant domain 6 of CD44 gene, Genecards ID (GCID): GC11P035139, UniProtKB/Swiss-Prot: P16070), CD45 (Genecards ID (GCID): GC01P198607, UniProtKB/Swiss-Prot: P08575), CDw52 (Genecards ID (GCID): GC01P026317, UniProtKB/Swiss-Prot: P31358), Fms-like tyrosine kinase 3 (FLT-3, CD135, Genecards ID (GCID): GC13M028003, UniProtKB/Swiss-Prot: P36888), c-Kit (CD117, Genecards ID (GCID): GC04P054657, UniProtKB/Swiss-Prot: P10721), CSFIR (Colony Stimulating Factor 1 Receptor, CD115, Genecards ID (GCID): GC05M150053, UniProtKB/Swiss-Prot: P07333), CD133 (Genecards ID (GCID): GC04M 015965, UniProtKB/Swiss-Prot: 043490), PDGFR-a (Platelet Derived Growth Factor Receptor Alpha, CD140a, Genecards ID (GCID): GC04P054229, UniProtKB/Swiss-Prot: P16234), PDGFR-β (Platelet Derived Growth Factor Receptor Beta, CD 140b, Genecards ID (GCID): GC05M150113, UniProtKB/Swiss-Prot: P09619), chondroitin sulfate proteoglycan 4 (CSPG4, melanoma-associated chondroitin sulfate proteoglycan, Genecards ID (GCID):

GC15M075674, UniProtKB/Swiss-Prot: Q6UVK1), EGFR (Epidermal Growth Factor Receptor, Genecards ID (GCID): GC07P055019, UniProtKB/Swiss-Prot: P00533), de2-7-EGFR (EGFRvIII, in which there is a deletion of the 801 bp encoded by exons 2-7, resulting in a truncated receptor (140 kDa) lacking 267 amino acid residues in the extracellular domain, with the insertion of a novel glycine residue at the site of the deletion. See, for example, Sugawa et al. Proc Natl Acad Sci USA. 1990 November; 87(21):8602-6 and Humphrey et al. Biochem Biophys Res Commun. 1991 Aug. 15; 178(3):1413-20), Folate binding protein (Genecards ID (GCID): GC11P072190, UniProtKB/Swiss-Prot: P15328), Her2neu (Genecards ID (GCID): GC17P039687, UniProtKB/Swiss-Prot: P04626), Her3 (Genecards ID (GCID): GC12P056376, UniProtKB/Swiss-Prot: P21860), PSMA (Prostate-Specific Membrane Antigen, Genecards ID (GCID): GC11M069030, UniProtKB/Swiss-Prot: Q04609), PSCA (Prostate Stem Cell Antigen, Genecards ID (GCID): GC08P142670,UniProtKB/Swiss-Prot: 043653), PSA (Prostate-Specific Antigen, Genecards ID (GCID): GC19P050854, UniProtKB/Swiss-Prot: P07288), Tumor-associated glycoprotein 72 (TAG-72, see, for example, Sheer et al. Cancer Res. 1988 Dec. 1; 48(23):6811-8), HLA-DR (Human Leukocyte Antigen-DR isotype, which is an αβ heterodimer. See, for example, Genecards ID (GCID): GC06P032439, UniProtKB/Swiss-Prot: P01903 for HLA Class II Histocompatibility Antigen, DR Alpha Chain; Genecards ID (GCID): GC06M032578, UniProtKB/Swiss-Prot: P01911 for HLA Class II Histocompatibility Antigen, DR-1 Beta Chain; Genecards ID (GCID): GC06Mn03715, UniProtKB/Swiss-Prot: P79483 for MHC Class II Antigen DR Beta 3 Chain; Genecards ID (GCID): GC06M003851, UniProtKB/Swiss-Prot: P13762 for HLA Class II Histocompatibility Antigen, DR Beta 4 Chain; and Genecards ID (GCID): GC06M046926, UniProtKB/Swiss-Prot: Q30154 for HLA Class II Histocompatibility Antigen, DR Beta 5 Chain), IGFR (Insulin Like Growth Factor 1 Receptor, Genecards ID (GCID): GC15P098648, UniProtKB/Swiss-Prot: P08069), IL3R (Interleukin 3 Receptor, see, for example, Genecards ID (GCID): GC0XP001336, UniProtKB/Swiss-Prot: P26951 for Interleukin 3 Receptor Subunit Alpha; and Genecards ID (GCID): GC22P036913, UniProtKB/Swiss-Prot: P32927 for Interleukin 3 Receptor Subunit Beta), fibroblast activating protein (FAP, Genecards ID (GCID): GC02M162170, UniProtKB/Swiss-Prot: Q12884), Carboanhydrase IX (MN/CA IX, Genecards ID (GCID): GC09P035673, UniProtKB/Swiss-Prot: Q16790), Carcinoembryonic antigen (CEA, Genecards ID (GCID): GC19P041709, UniProtKB/Swiss-Prot: P06731), EpCAM (Epithelial Cell Adhesion Molecule, Genecards ID (GCID): GC02P047345, UniProtKB/Swiss-Prot: 16422), CDCP 1 (CUB Domain Containing Protein 1, Genecards ID (GCID): GC03M 045082, UniProtKB/Swiss-Prot: Q9H5V8), DERL1 (Derlin1 or Derlin 1, Genecards ID (GCID): GC08M123013, UniProtKB/Swiss-Prot: Q9BUN8), Tenascin (for example, Genecards ID (GCID): GC06M046900, UniProtKB/Swiss-Prot: P22105 for Tenascin XB; Genecards ID (GCID): GC09M115019, UniProtKB/Swiss-Prot: P24821 for Tenascin C; Genecards ID (GCID): GC01M175291, UniProtKB/Swiss-Prot: Q92752 for Tenascin R, Genecards ID (GCID): GC01P175067, UniProtKB/Swiss-Prot: Q9UQP3 for Tenascin N; Genecards ID (GCID): GC06M046903, UniProtKB/Swiss-Prot: Q16473 for Tenascin XA), frizzled 1-10 (see, for example, Genecards ID (GCID): GC07P091264, UniProtKB/Swiss-Prot: Q9UP38 for frizzled 1 or FZD 1; Genecards ID (GCID): GC17P 044557, UniProtKB/Swiss-Prot: Q14332 for frizzled 2 or FZD2; Genecards ID (GCID): GC08P028494, UniProtKB/Swiss-Prot: Q9NPG1 for frizzled or FZD3; Genecards ID (GCID): GC11M086945, UniProtKB/Swiss-Prot: Q9ULV 1 for frizzled 4 or FZD 4; Genecards ID (GCID): GC02M207762, UniProtKB/Swiss-Prot: Q13467 for frizzled 5 or FZD5; Genecards ID (GCID): GC08P 103298, UniProtKB/Swiss-Prot: 060353 for frizzled 6 or FZD 6; Genecards ID (GCID): GC02P 202034, UniProtKB/Swiss-Prot: 075084 for frizzled 7 or FZD 7;

Genecards ID (GCID): GC10M035638, UniProtKB/Swiss-Prot: Q9H461 for frizzled 8 or FZD8; Genecards ID (GCID):

GC07P 073433, UniProtKB/Swiss-Prot: 000144 for frizzled 9 or FZD 9; Genecards ID (GCID): GC12P130162, UniProtKB/Swiss-Prot: Q9ULW2 for frizzled 10 or FZD10), VEGFR2 (Vascular Endothelial Growth Factor Receptor 2, or KDR/FLK1, Genecards ID (GCID): GC04M055078, UniProtKB/Swiss-Prot: P35968), VEGFR3 (Vascular Endothelial Growth Factor Receptor 3, FLT4, or CD309, Genecards ID (GCID): GC05M180607, UniProtKB/Swiss-Prot: P35916), ENG (Endoglin, Genecards ID (GCID): GC09M127815, UniProtKB/Swiss-Prot: P17813), CLEC14 (see, for example, Genecards ID (GCID):

GC14M038254, UniProtKB/Swiss-Prot: Q86T13 for C-Type Lectin Domain Containing 14A or CLEC14A), Tem1-8 (see, for example, Genecards ID (GCID): GC11M066314, UniProtKB/Swiss-Prot: Q9HCU0 for Tumor Endothelial Marker 1 or TEM1; Genecards ID (GCID): GC22P035540, UniProtKB/Swiss-Prot: Q96D21 for Tumor Endothelial Marker 2 or TEM2; Genecards ID (GCID): GC17M039063, UniProtKB/Swiss-Prot: 8QIUK5 for Tumor Endothelial Marker 3, TEM3, Tumor Endothelial Marker 7, or TEM7; Genecards ID (GCID): GC11P073306, UniProtKB/Swiss-Prot: Q96PE2 for Tumor Endothelial Marker 4 or TEM4; Genecards ID (GCID): GC08P037785, UniProtKB/Swiss-Prot: Q96PE1 for Tumor Endothelial Marker 5 or TEM5; Genecards ID (GCID): GC07M047281, UniProtKB/Swiss-Prot: Q68CZ2 for Tumor Endothelial Marker 6 or TEM6; or Genecards ID (GCID): GC02P068977, UniProtKB/Swiss-Prot: Q9H6X2 for Tumor Endothelial Marker 8 or TEM8), or Tie2 (TEK Receptor Tyrosine Kinase, CD202b, Genecards ID (GCID): GC09P027109, UniProtKB/Swiss-Prot: Q02763). Each of the Genecard and UniProt webpages following the identified reference numbers is incorporated by references herein in its entirety.

As used herein, the term “embryonic antigen” refers to an antigen expressed by an embryonic cells. Some cancer antigens are embryonic antigens, whose expression is normally restricted to fetal cells but which are abnormally expressed in adult cells that have undergone malignant transformation. The gene encoding the embryonic antigen is silent in normal adult tissues but reactivated in cancer cells. An example of an embryonic antigen is carcinoembryonic antigen (CEA), which is normally expressed only in the liver, intestines and pancreas of the human fetus but is highly associated with colon, breast and ovarian cancers in the adult. Similarly, alpha-fetoprotein (AFP) is normally expressed only in fetal liver and yolk sac cells but is strongly linked to liver and testicular cancers in the adult. In some embodiments, the embryonic antigen comprises, or alternatively consists essentially of, or yet further consists of one or more of: Ephrin Type-A Receptor 2 (EphA2, Genecards ID (GCID): GC01M016124, UniProtKB/Swiss-Prot: P29317), Interleukin 13 Receptor Subunit Alpha 2 (IL13Ra2, Genecards ID (GCID): GC0XM115003, UniProtKB/Swiss-Prot: Q14627) homodimers, carcinoembryonic antigen (CEA, Genecards ID (GCID): GC19P041709,UniProtKB/Swiss-Prot: P06731), or heat shock protein (hsp) gp96 (HSP96, Genecards ID (GCID): GC12P103930, UniProtKB/Swiss-Prot: P14625). Each of the Genecard and UniProt webpages following the identified reference numbers is incorporated by references herein in its entirety.

As used herein, the term “epithelial tumor antigen” or “ETA” refers to an antigen expression by an epithelial cancer cell. Non-limiting examples of ETA include Carcinoembryonic antigen (CEA, Genecards ID (GCID): GC19P041709, UniProtKB/Swiss-Prot: P06731), EpCAM (Genecards ID (GCID): GC02P047345, UniProtKB/Swiss-Prot: 16422), PSA (Prostate-Specific Antigen, Genecards ID (GCID): GC19P050854,UniProtKB/Swiss-Prot: P07288), HER-2/neu (Genecards ID (GCID): GC17P039687, UniProtKB/Swiss-Prot: P04626) and MUC1 (Genecards ID (GCID): GC01M155185, UniProtKB/Swiss-Prot: 15941). Each of the Genecard and UniProt webpages following the identified reference numbers is incorporated by references herein in its entirety.

As used herein, the term melanoma-associated antigen (MAGE) refers to a mammalian member of the MAGE (melanoma-associated antigen) gene family sharing a stretch of about 200 amino acids which has been named the MAGE conserved domain. Non-limiting examples of MAGE include, MAGE-B1 (Genecards ID (GCID): GC0XP030244, UniProtKB/Swiss-Prot: P43366), MAGEA1 (Genecards ID (GCID); GC0XP153179, UniProtKB/Swiss-Prot: P43355), MAGEA10 (Genecards ID (GCID): GC0XM152133, UniProtKB/Swiss-Prot: P43363), MAGEA11 (Genecards ID (GCID): GC0XP149688, UniProtKB/Swiss-Prot: P43364), MAGEA12 (Genecards ID (GCID): GC0XP152733, UniProtKB/Swiss-Prot: P43365), MAGEA2B (Genecards ID (GCID): GC0XP152714, UniProtKB/Swiss-Prot: P43356), MAGEA3 (Genecards ID (GCID): GC0XP152698, UniProtKB/Swiss-Prot: P43357), MAGEA4 (Genecards ID (GCID): GC0XP151912, UniProtKB/Swiss-Prot: P43358), MAGEA6 (Genecards ID (GCID): GC0XM152766, UniProtKB/Swiss-Prot: P43360), MAGEA8 (Genecards ID (GCID): GC0XP149881, UniProtKB/Swiss-Prot: P43361), MAGEA9 (Genecards ID (GCID): GC0XP149781, UniProtKB/Swiss-Prot: P43362), MAGEB1 (Genecards ID (GCID): GC0XP030244, UniProtKB/Swiss-Prot: 43366), MAGEB 10 (Genecards ID (GCID): GC0XP 027808, UniProtKB/Swiss-Prot: Q96LZ2), MAGEB16 (Genecards ID (GCID): GC0XP035816, UniProtKB/Swiss-Prot: 2A368), MAGEB18 (Genecards ID (GCID): GC0XP026138, UniProtKB/Swiss-Prot: Q96M61), MAGEB2 (Genecards ID (GCID): GC0XP030215, UniProtKB/Swiss-Prot: 015479), MAGEB 3 (Genecards ID (GCID): GC0XP 030230, UniProtKB/Swiss-Prot: 015480), MAGEB 4 (Genecards ID (GCID): GCOXP 030241, UniProtKB/Swiss-Prot: 015481), MAGEB 5 (Genecards ID (GCID): GC0XP026229, UniProtKB/Swiss-Prot: Q9BZ81), MAGEB6 (Genecards ID (GCID):, UniProtKB/Swiss-Prot:), MAGEB6B (Genecards ID (GCID): GC0XP026192, UniProtKB/Swiss-Prot: Q8N7X4), MAGEC1 (Genecards ID (GCID): GC0XP141905, UniProtKB/Swiss-Prot: O60732), MAGEC2 (Genecards ID (GCID): GC0XM142202, UniProtKB/Swiss-Prot: Q9UBF1), MAGEC3(Genecards ID (GCID): GC0XP141838, UniProtKB/Swiss-Prot: Q8TD91), MAGED1 (Genecards ID (GCID): GCOXP051803, UniProtKB/Swiss-Prot: Q9Y5V3), MAGED2 (Genecards ID (GCID): GC0XP054807, UniProtKB/Swiss-Prot: Q9UNF1), MAGED4 (Genecards ID (GCID): GC0XP052184, UniProtKB/Swiss-Prot: Q96JG8), MAGEE1 (Genecards ID (GCID): GC0XP076427, UniProtKB/Swiss-Prot: 9HCI5), MAGEE2 (Genecards ID (GCID): GC0XM075782, UniProtKB/Swiss-Prot: Q8TD90), MAGEF1 (Genecards ID (GCID): GC03M184710, UniProtKB/Swiss-Prot: 9HAY2), MAGEH1 (Genecards ID (GCID): GC0XP055452, UniProtKB/Swiss-Prot: Q9H213), MAGEL2 (Genecards ID (GCID): GC15M023643, UniProtKB/Swiss-Prot: Q9UJ55), NDN (Genecards ID (GCID): GC15M023686, UniProtKB/Swiss-Prot: Q99608), or NDNL2 (Genecards ID (GCID): GC15M029269, UniProtKB/Swiss-Prot: Q96MG7). Each of the Genecard and UniProt webpages following the identified reference numbers is incorporated by references herein in its entirety.

As used herein, an abnormal product of ras refers to a gene product encoded by a mutated RAS gene. It has been known for more than three decades that about a third of all human cancers, including a high percentage of pancreatic, lung, and colorectal cancers, are driven by mutations in RAS genes. The main members of the RAS gene family, including but not limited to KRAS (Genecards ID (GCID): GC12M025204, UniProtKB/Swiss-Prot: P01116), HRAS (Genecards ID (GCID): GC11M001078, UniProtKB/Swiss-Prot: P01112), and NRAS (Genecards ID (GCID): GC01M114704, UniProtKB/Swiss-Prot: P01111), encode proteins that have a pivotal cytoplasmic role in cell signaling. When RAS genes are mutated, cells grow uncontrollably and evade death signals. For example, NRAS mutations which change AA 12, 13 or 61 activate the potential of Ras to transform cultured cells and are implicated in a variety of human tumors; the amino acid positions that account for the overwhelming majority of the KRAS mutations are G12, G13 and Q61; and HRAS mutations which change positions 12, 13 or 61 activate the potential of HRAS to transform cultured cells and are implicated in a variety of human tumors. RAS mutations also make cells resistant to some available cancer therapies. Each of the Genecard and UniProt webpages following the identified reference numbers is incorporated by references herein in its entirety.

In one embodiment, an “immunogenic fragment” refers to a portion of an oligopeptide, polypeptide or protein that is immunogenic and elicits a protective immune response when administered to a subject.

In one embodiment, “immunogenicity” or “immunogenic” is used herein to refer to the innate ability of a protein, peptide, nucleic acid, antigen or organism to elicit an immune response in an animal when the protein, peptide, nucleic acid, antigen or organism is administered to the animal. Thus, “enhancing the immunogenicity” in one embodiment, refers to increasing the ability of a protein, peptide, nucleic acid, antigen or organism to elicit an immune response in an animal when the protein, peptide, nucleic acid, antigen or organism is administered to an animal. The increased ability of a protein, peptide, nucleic acid, antigen or organism to elicit an immune response can be measured by, in one embodiment, a greater number of antibodies to a protein, peptide, nucleic acid, antigen or organism, a greater diversity of antibodies to an antigen or organism, a greater number of T-cells specific for a protein, peptide, nucleic acid, antigen or organism, a greater cytotoxic or helper T-cell response to a protein, peptide, nucleic acid, antigen or organism, and the like.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

### Modes for Carrying out the Disclosure

Clinical grade current Good Manufacturing Product (cGMP) exists for several oncolytic viruses (OVs). The time and cost required for cGMP production, validation, biotoxicity testing and regulatory approval is prohibitive and, in some cases, has taken up to a decade for some vectors (e.g., M032, or MB108 (C134)). During the time required for such validation and approval, technological advances occur that investigators are unable to incorporate into their vectors to improve activity because genetic modifications would lead to restarting the production and approval process and delaying entry into human clinical trial. To test whether viruses with synergistic or complementing activity can be mixed together and administered to maximize their therapeutic effect, a series of oncolytic virus based in vivo studies were performed in tumor bearing mice to determine if virus combinations were effective and superior to the individual therapies alone. The results showed that using rational combinations by mixing together different viruses is more effective than equivalent doses of either virus alone. Without wishing to be bound by the theory, OV combinations using existing cGMP stocks can be employed effectively thus saving the expense and time required in the re-engineering, production and validation phase required for cGMP product development. The new combinations requires regulatory approval and safety but this allows a more rapid and cost effective approach to clinical translation using existing stocks of virus. This patent applies to this method of combining viruses to improve clinical outcome.

Additionally, without wishing to be bound by the theory, the OV combination shows immune response characteristics (such as, the tumor-infiltrating immune cells) different compared to those treated with the individual therapy, thus impacting immune function against the tumor.

It was also found that, for these mixing studies, because the cytokine is altering the tumor microenvironment, the viruses in the combination can complement one another. In some embodiments, two or more different infected cells can work together, for example, in trans expression. In contrast, a single virus producing all or both of the genes expressed by the virus combination from a single infected cell, for example, in cis expression, can be used, but not necessary.

The composition and method as disclosed herein reduce the time and cost involved in engineering, manufacturing, production and validation for OV development allowing clinical grade OV mixing for more rapid OV translation to patients.

### Oncolytic Virus

In one aspect, provided is an oncolytic virus expressing: an anti-cancer cytokine, or an anti-cancer transcriptional regulator, or both, and a cancer antigen, or an immune cell engager specifically recognizing and binding to the cancer antigen, or both.

In some embodiments, the oncolytic virus is one single polycistronic oncolytic virus expressing: an anti-cancer cytokine, or an anti-cancer transcriptional regulator, or both; and a cancer antigen, or an immune cell engager specifically recognizing and binding to the cancer antigen, or both.

In some embodiments, provided are more than one (such as two, or three, or four, or five, or six, or seven, or eight, or nine, or ten, or more) oncolytic viruses. In further embodiments, the more than one oncolytic virus together express one or more of: an anti-cancer cytokine, an anti-cancer transcriptional regulator, a cancer antigen, or an immune cell engager specifically recognizing and binding to the cancer antigen. In yet further embodiments, the more than one oncolytic virus comprise, or alternatively consist essentially of, or yet further consist of a first oncolytic virus expressing an anti-cancer cytokine, or an anti-cancer transcriptional regulator, or both and a second oncolytic virus expressing a cancer antigen, or an immune cell engager specifically recognizing and binding to the cancer antigen, or both. In yet further embodiments, the first oncolytic virus or the second oncolytic virus or both are polycistronic (such as bicistronic) oncolytic viruses. For example, the first oncolytic virus can be polycistronic (such as bicistronic) and express an anti-cancer cytokine and an anti-cancer transcriptional regulator. In some embodiments, the first oncolytic virus can be polycistronic (such as bicistronic) and express two or more anti-cancer cytokines. In some embodiments, the second oncolytic virus can be polycistronic (such as bicistronic) and express a cancer antigen, and an immune cell engager specifically recognizing and binding to the cancer antigen. In some embodiments, the second oncolytic virus can be polycistronic (such as bicistronic) and express two or more cancer antigens.

In some embodiments, provided are more than two (such as three, or four, or five, or six, or seven, or eight, or nine, or ten, or more) oncolytic viruses. In further embodiments, the more than two oncolytic viruses comprise, or alternatively consist essentially of, or yet further consist of a first oncolytic virus, a second oncolytic virus and a third oncolytic virus, each of which can be monocistronic or polycistronic (such as bicistronic). In yet further embodiments, the more than two oncolytic viruses together express one or more of: an anti-cancer cytokine, an anti-cancer transcriptional regulator, a cancer antigen, or an immune cell engager specifically recognizing and binding to the cancer antigen.

In some embodiments, an oncolytic virus as disclosed herein, including those comprised by a composition or a kit as disclosed herein, those used by a method as disclosed herein, or both, is or is derived from an adenovirus, an adeno-associated virus (AAV), a reovirus, a measles, a herpes simplex virus (HSV), a Newcastle disease virus (NDV), a vesicular stomatitis virus (VSV), a poliovirus (PV), an ECHO-7 (RIGVIR), a semliki forest virus (SFV), a maraba virus, a coxsackievirus A21, a senecavirus, a Pox virus, a retrovirus, a vesiculovirus, a parvovirus, or an influenza.

In some embodiments, the Pox virus comprises, or alternatively consists essentially of, or yet further consists of one or more of: a vaccinia virus (VACV), a cowpox, a myxoma virus, or a racoonpox virus (RPV), squirrelpox virus, or yaba monkey tumor virus.

In some embodiments, the HSV comprises, or alternatively consists essentially of, or yet further consists of either or both of HSV-1 or HSV-2.

In some embodiments, the retrovirus comprises, or alternatively consists of, or yet further consists of gamma-retrovirus or lentivirus.

In some embodiments, the vesiculovirus comprises, or alternatively consists of, or yet further consists of either or both of vesicular stomatitis virus or maraba virus.

In some embodiments, the oncolytic virus comprises a polynucleotide encoding one or more of: an anti-cancer cytokine, an anti-cancer transcriptional regulator, a cancer antigen, or an immune cell engager specifically recognizing and binding to the cancer antigen as disclosed herein. In some embodiments, the oncolytic virus comprises a vector genome encoding one or more of: an anti-cancer cytokine, an anti-cancer transcriptional regulator, a cancer antigen, or an immune cell engager specifically recognizing and binding to the cancer antigen as disclosed herein. In further embodiments, the polynucleotide or the vector genome further comprises a regulatory sequence, such as a promoter or an enhancer, directing the expression of the one or more of: an anti-cancer cytokine, an anti-cancer transcriptional regulator, a cancer antigen, or an immune cell engager specifically recognizing and binding to the cancer antigen as disclosed herein.

Another suitable oncolytic virus can be selected by one of skill in the art and engineered to express one or more of: an anti-cancer cytokine, an anti-cancer transcriptional regulator, a cancer antigen, or an immune cell engager specifically recognizing and binding to the cancer antigen as disclosed herein.

In one aspect, provided herein is a system for generating an oncolytic virus as disclosed herein. In some embodiments, the system comprises, or alternatively consists essentially of, or yet further consists of a first vector encoding the vector genome of the oncolytic virus. In further embodiments, the vector genome expresses one or more of: an anti-cancer cytokine, an anti-cancer transcriptional regulator, a cancer antigen, or an immune cell engager specifically recognizing and binding to the cancer antigen as disclosed herein, optionally the expression of which is under the direction of a regulatory sequence, such as a promoter or an enhancer. In yet further embodiments, the first vector is replication-defective, such as lacking one or more viral gene critical for packaging the vector genome into infectious viral particles. In some embodiments, the first vector is a non-viral vector, such as a plasmid, or a viral vector. In some embodiments, the system further comprises a second vector providing the lacked viral gene critical for packaging the vector genome into infectious viral particles. In further embodiments, the second vector is a virus, and thus referred to herein as a helper virus. In some embodiments, the system further comprises a host cell. In further embodiments, the host cell is introduced (such as transfected or transduced) with the first vector and optionally the second vector, the vector genome is packaged into infectious viral particles in the host cell, and thus the oncolytic virus is generated.

In a further aspect, provided herein is a method for generating an oncolytic virus. The method comprises, or alternatively consists essentially of, or yet further consists of culturing the host cell comprising the first vector and optionally the second vector under conditions suitable for packing the oncolytic virus, and collecting the oncolytic virus generated. In some embodiments, the method further comprises introducing the first vector into the host cell prior to the culturing step, optionally by transfection or transduction. In further embodiments, the method further comprises introducing the second vector into the host cell prior to the culturing step, optionally by transfection or transduction. In yet further embodiments, the method further comprises purifying the collected oncolytic virus, optionally by one or more of: ultracentrifugation, chromatography, precipitation, or nanofiltration.

In one aspect, provided herein is a host cell comprising an oncolytic virus as disclosed herein. In some embodiments, the host cell is suitable for replicating the oncolytic virus, such as a cancer cell.

In a further aspect, provided is a method for producing an oncolytic virus as disclosed herein. The method comprises, or alternatively consists essentially of, or yet further consists of culturing the host cell comprising the oncolytic virus under conditions suitable for producing the oncolytic virus, and collecting the oncolytic virus generated. In some embodiments, the method further comprises introducing the oncolytic virus into the host cell prior to the culturing step, optionally by transfection or transduction. In further embodiments, the method further comprises purifying the collected oncolytic virus, optionally by one or more of: ultracentrifugation, chromatography, precipitation, or nanofiltration. In one example of the method, master and working seed stocks can be prepared under Good Manufacturing Practice (GMP) conditions. In some instances, cells are plated on large surface area flasks, grown to near confluency, and infected at selected multiplicity of infection (MOI). The produced virus can then be purified. In some cases, cells are harvested and intracellular virus is released by mechanical disruption. In some embodiments, cell debris is removed by large-pore depth filtration and/or host cell DNA is digested with an endonuclease. In some cases, virus particles are subsequently purified and concentrated by tangential-flow filtration, followed by diafiltration. The resulting concentrated virus can formulated by dilution with a buffer containing one or more stabilizers, filled into vials, and lyophilized. Compositions and formulations can be stored for later use. In some embodiments, a lyophilized virus is reconstituted by addition of one or more diluents.

In some embodiments, the vector as disclosed herein, such as the first vector or the second vector or both, is a non-viral vector, for example, a plasmid. In some embodiments, the vector as disclosed herein, such as the first vector or the second vector or both, is a viral vector. In further embodiments, the viral vector is the one which the oncolytic virus is derived from.

In some embodiments, the host cell is a prokaryotic cell or a eukaryotic cell. In some embodiments, the host cell is a cell line such as a mammalian cell line, an insect cell line, or a plant cell line, optionally infected with an oncolytic virus described herein to produce one or more viruses. Exemplary mammalian cell lines include: 293A cell line, 293 FT cell line, 293F cells, 293 H cells, CHO DG44 cells, CHO-S cells, CHO-K1 cells, Expi293F™ cells, Flp-In™ T-REX™ 293 cell line, Flp-In™-293 cell line, Flp-In™-3T3 cell line, Fip-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REX™ Jurkat cell line, Per C6 cells, T-REX™-293 cell line, T-REX™-CHO cell line, T-REx™-HeLa cell line, 3T6, A549, A9, AtT-20, BALB/3T3, BHK-21, BHL-100, BT, Caco-2, Chang, Clone 9, Clone M-3, COS-1, COS-3, COS-7, CRFK, CV-1, D-17, Daudi, GHI, GH3, H9, HaK, HCT-15, HEp-2, HL-60, HT-1080, HT-29, HUVEC, I-10, IM-9, JEG-2, Jensen, K-562, KB, KG-1, L2, LLC-WRC 256, McCoy, MCF 7, VERO, WI-38, WISH, XC, or Y-1. Exemplary insect cell lines include Drosophila S2 cells, Sf9 cells, Sf21 cells, High FiveT cells, or expresSF+® cells. Exemplary plant cell lines include algae cells such as for example Phaeocystis pouchetii.

### Compositions

In one aspect, provided is a composition comprising, or alternatively consisting essentially of, or yet further consisting of an oncolytic virus as disclosed herein. In some embodiments, the composition comprises, or alternatively consists essentially of, or yet further consists of the single polycistronic oncolytic virus as disclosed herein. In some embodiments, the composition comprises, or alternatively consists essentially of, or yet further consists of the more than one oncolytic virus as disclosed herein. In further embodiments, the composition comprises, or alternatively consists essentially of, or yet further consists of the more than two oncolytic virus as disclosed herein.

In a further aspect, provided is a composition comprising, or alternatively consisting essentially of, or yet further consisting of a first oncolytic virus expressing an anti-cancer cytokine, or an anti-cancer transcriptional regulator, or both; and a second oncolytic virus expressing one or more of: a cancer antigen, an immune cell engager specifically recognizing and binding to the cancer antigen, or an anti-cancer cytokine that is not the one expressed by the first oncolytic virus.

In another aspect, provided is a composition comprising, or alternatively consisting essentially of, or yet further consisting of an oncolytic virus expressing an anti-cancer cytokine, or an anti-cancer transcriptional regulator, or both; and an immune cell specifically recognizing and binding to a cancer antigen, with the proviso that (i) the anti-cancer cytokine does not comprise any one of interleukin 15 (IL-15), RANTES, interleukin 2 (IL-2), Tumor Necrosis Factor α (TNFα), or C-X-C Motif Chemokine Ligand 11 (CXCL11), or (ii) the immune cell does not comprise a Chimeric Antigen Receptor (CAR) T cell, or both (i) and (ii). In some embodiments, the anti-cancer cytokine comprises, or alternatively consists essentially of, or yet further consists of any one or more of: interleukin 12 (IL-12), interleukin 21 (IL-21), interleukin 27 (IL-27), or C-X-C Motif Chemokine Ligand 10 (CXCL10).

In some embodiments, the composition comprises, or alternatively consists essentially of, or yet further consists of a first oncolytic virus expressing IL-12 or an equivalent thereof and a second oncolytic virus expressing Ephrin Type-A Receptor 2 (EphA2) or an immunogenic fragment thereof.

In some embodiments, the composition comprises, or alternatively consists essentially of, or yet further consists of a first oncolytic virus expressing IL-12 or an equivalent thereof and a second oncolytic virus expressing a bispecific T-cell engager that binds to CD3 and EphA2 or an immunogenic fragment thereof.

In some embodiments, the composition comprises, or alternatively consists essentially of, or yet further consists of a first oncolytic virus expressing IL-21 or an equivalent thereof and a second oncolytic virus expressing C-X-C Motif Chemokine Ligand 10 (CXCL10) or an equivalent thereof.

In some embodiments, the composition comprises, or alternatively consists essentially of, or yet further consists of a first oncolytic virus expressing IL-21 and CXCL10 or an equivalent of each thereof, and an immune cell comprising, or alternatively consisting essentially of, or yet further consisting of a splenocyte that specifically recognizes and binds EphA2.

In some embodiments, the composition further comprises a carrier, optionally a pharmaceutical acceptable carrier. In further embodiments relating to more than one compositions, each of the compositions can further comprise a carrier, optionally a pharmaceutically acceptable carrier. Such carrier or pharmaceutically acceptable carrier can be selected by one of skill in the art based on the active agent (such as the oncolytic virus(es) as disclosed herein) in the composition, the route of administration, the storage condition of the composition (such as the storage temperature, or the storage vial), the disease to be treated, and the subject to be treated.

### Methods of Treatment

In one aspect, provided is a method of treating a subject having a cancer or suspect of having a cancer.

In some embodiments, the method comprises, or alternatively consists of, or yet further consists of administering, for example an effective amount of, an oncolytic virus as disclosed herein to the subject. In further embodiments, the method comprises, or alternatively consists of, or yet further consists of administering, for example an effective amount of, more than one oncolytic virus as disclosed herein to the subject. In yet further embodiments, the more than one oncolytic virus can be administered in one composition or different compositions. In some embodiments, the more than one oncolytic virus are administered in different compositions concurrently. In some embodiments, the more than one oncolytic virus are administered in different compositions sequentially. In further embodiments, each of the two sequential administrations can be about 1 hour apart, about 2 hours apart, about 3 hours apart, about 4 hours apart, about 5 hours apart, about 6 hours apart, about 7 hours apart, about 8 hours apart, about 9 hours apart, about 10 hours apart, about 11 hours apart, about 12 hours part, about 1 day apart, about 2 days apart, about 3 days apart, about 4 days apart, about 5 days apart, about 6 days apart, about 7 days apart, about 2 weeks apart, about 3 weeks apart, about 4 weeks apart, about 5 weeks part, about 6 weeks apart, about 7 weeks apart, about 2 months apart, or longer. In some embodiments, the same or different routes of administrations can be used for the sequential administrations.

In some embodiments, the method comprises, or alternatively consists of, or yet further consists of administering, for example an effective amount of, a composition as disclosed herein to the subject. In further embodiments, more than one composition as disclosed herein can be administered to the subject, concurrently or sequentially. In yet further embodiments, each of the two sequential administrations can be about 1 hour apart, about 2 hours apart, about 3 hours apart, about 4 hours apart, about 5 hours apart, about 6 hours apart, about 7 hours apart, about 8 hours apart, about 9 hours apart, about 10 hours apart, about 11 hours apart, about 12 hours part, about 1 day apart, about 2 days apart, about 3 days apart, about 4 days apart, about 5 days apart, about 6 days apart, about 7 days apart, about 2 weeks apart, about 3 weeks apart, about 4 weeks apart, about 5 weeks part, about 6 weeks apart, about 7 weeks apart, about 2 months apart, or longer. In some embodiments, the same or different routes of administrations can be used for the sequential administrations.

In some embodiments, the method comprises, or alternatively consists of, or yet further consists of administering, for example an effective amount of, the first and second compositions of a kit as disclosed herein to the subject. In further embodiments, the first composition and the second composition are administered to the subject concurrently or sequentially. In the embodiments where the first composition and the second composition are administered sequentially, the two administrations are about 1 hour apart, about 2 hours apart, about 3 hours apart, about 4 hours apart, about 5 hours apart, about 6 hours apart, about 7 hours apart, about 8 hours apart, about 9 hours apart, about 10 hours apart, about 11 hours apart, about 12 hours part, about 1 day apart, about 2 days apart, about 3 days apart, about 4 days apart, about 5 days apart, about 6 days apart, about 7 days apart, about 2 weeks apart, about 3 weeks apart, about 4 weeks apart, about 5 weeks part, about 6 weeks apart, about 7 weeks apart, about 2 months apart, or longer. In some embodiments, the same or different routes of administrations can be used for the sequential administrations.

In some embodiments where at least one oncolytic virus expresses an anti-cancer antigen or an immune cell engager specifically recognizing and binding the cancer antigen or both, the cancer to be treated expresses the cancer antigen, such as EphA2. In some embodiments where at least one oncolytic virus expresses an anti-cancer antigen or an immune cell engager specifically recognizing and binding the cancer antigen or both, the subject comprises a cancer expressing the cancer antigen, such as EphA2.

In some embodiments, the subject is a mammal. In further embodiments, the subject is a human. Additionally, or alternatively, the subject is an adult.

In some embodiments, the method is used as a first line therapy, or a second line therapy, or a third line therapy, or a fourth line therapy. In some embodiment, the method can be combined with another anti-cancer therapy, such as a tumor resection, a chemotherapy, a cell comprising a Chimeric Antigen Receptor (CAR) such as a CAR T-cell or CAR-NK cell, a radiation therapy, or an immunotherapy.

### Kits

In one aspect, provided is a kit comprising, or alternatively consisting essentially of, or yet further consisting of instructions for use; a first composition comprising a first oncolytic virus expressing an anti-cancer cytokine, or an anti-cancer transcriptional regulator; and a second composition comprising a second oncolytic virus expressing one or more of: a cancer antigen, an immune cell engager specifically recognizing and binding the cancer antigen, or an anti-cancer cytokine that is not the one expressed by the first oncolytic virus.

In some embodiments, the first composition further comprising a carrier, such as a pharmaceutically acceptable carrier. Additionally or alternatively, the second composition further comprising a carrier, such as a pharmaceutically acceptable carrier.

In some embodiments, the kit is for use in treating a cancer in a subject in need thereof, such as in a method as disclosed herein.

In a further aspect, provided is a kit for use in a method as disclosed herein. The kit comprises, or alternatively consists essentially of, or yet further consists of instructions for use and one or more of: an oncolytic virus as disclosed herein or a composition as disclosed herein.

Other components can be further included in the kit, such as a dilute of the oncolytic virus, a device for administration (such as a syringe), or a container suitable for storing and shipping the oncolytic virus.

### Cancer and Cancer Antigens

In some embodiments, the cancer antigen as referred to herein comprises, or alternatively consists essentially of, or yet further consists of an embryonic antigen. In further embodiments, the embryonic antigen comprises, or alternatively consists essentially of, or yet further consists of one or more of: Ephrin Type-A Receptor 2 (EphA2), Interleukin 13 Receptor Subunit Alpha 2 (IL13Ra2) homodimers, carcinoembryonic antigen (CEA), or heat shock protein (hsp) gp96 (HSP96).

In some embodiments, the cancer antigen as referred to herein comprises, or alternatively consists essentially of, or yet further consists of one or more of: an embryonic antigen, alphafetoprotein (AFP), CA-125 (Mucin 16, Cell Surface Associated), MUC-1 (Mucin 1, Cell Surface Associated), Epithelial tumor antigen (ETA), Tyrosinase, Melanoma-associated antigen (MAGE), abnormal products of ras, p53, CD10, CD19, CD20, CD21, CD22, CD25, CD30, CD33, CD34, CD37, CD44v6, CD45, CDw52, Fms-like tyrosine kinase 3 (FLT-3, CD135), c-Kit (CD117), CSFIR (Colony Stimulating Factor |Receptor, CD115), CD133, PDGFR-α (CD140a), PDGFR-β (CD 140b), chondroitin sulfate proteoglycan 4 (CSPG4, melanoma-associated chondroitin sulfate proteoglycan), EGFR (Epidermal Growth Factor Receptor), de2-7-EGFR (EGFRvIII), Folate binding protein, Her2neu, Her3, PSMA (Prostate-Specific Membrane Antigen), PSCA (Prostate Stem Cell Antigen), PSA (Prostate-Specific Antigen), TAG-72, HLA-DR, IGFR, IL3R, fibroblast activating protein (FAP), Carboanhydrase IX (MN/CA IX), Carcinoembryonic antigen (CEA), EpCAM, CDCP1, Derlin1, Tenascin, frizzled 1-10, VEGFR2 (KDR/FLK1), VEGFR3 (FLT4, CD309), Endoglin, CLEC14, Tem1-8, or Tie2.

In some embodiments, the cancer antigen as referred to herein comprises, or alternatively consists essentially of, or yet further consists of Ephrin Type-A Receptor 2 (EphA2). In some embodiments, the cancer antigen as referred to herein comprises, or alternatively consists essentially of, or yet further consists of carcinoembryonic antigen (CEA). In some embodiments, the cancer antigen as referred to herein comprises, or alternatively consists essentially of, or yet further consists of an Interleukin 13 Receptor Subunit Alpha 2 (IL13Ra2) homodimer. In some embodiments, the cancer antigen as referred to herein comprises, or alternatively consists essentially of, or yet further consists of heat shock protein (hsp) gp96 (HSP96).

### Anti-Cancer Cytokine and Anti-Cancer Transcriptional Regulator

In some embodiments, the anti-cancer cytokine comprises, or alternatively consists essentially of, or yet further consists of one or more of: Interleukin 12 (IL-12), Interleukin 18 (IL-18), Interleukin 15 (IL-15), Interleukin 21 (IL-21), Interleukin 27 (IL-27), C-X-C Motif Chemokine Ligand 9 (CXCL9), C-X-C Motif Chemokine Ligand 10 (CXCL10), C-X-C Motif Chemokine Ligand 11 (CXCL11), Interferon α (IFNα), Type I interferon (IFNI), Type II interferon (IFNII), Interferon γ (IFNγ), Interleukin 2 (IL-2), Interleukin 5 (IL-5), Tumor Necrosis Factor (TNF), Interferon β (IFNβ), Interleukin 18 (IL-18), CD40 Ligand (CD40L), Colony Stimulating Factor 2 (CSF2), or an equivalent of each thereof.

In some embodiments, the anti-cancer cytokine comprises, or alternatively consists essentially of, or yet further consists of IL-12 or an equivalent thereof. In some embodiments, the anti-cancer cytokine comprises, or alternatively consists essentially of, or yet further consists of IL-21 or an equivalent thereof. In some embodiments, the anti-cancer cytokine comprises, or alternatively consists essentially of, or yet further consists of CXCL10 or an equivalent thereof. In some embodiments, the anti-cancer cytokine comprises, or alternatively consists essentially of, or yet further consists of IL-21 and CXCL10, or an equivalent of each thereof. In some embodiments, the anti-cancer cytokine comprises, or alternatively consists essentially of, or yet further consists of IL-27 or an equivalent thereof.

In some embodiments, the anti-cancer transcriptional regulator activates one or more of: an anti-cancer cytokine, Interferon Regulatory Factor 7 (IRF7), Interferon Alpha And Beta Receptor Subunit 1 (IFNAR1), T cell Receptor (TCR), Toll-like receptor 3 (TLR3), Interleukin 7 Receptor (IL7R), CD40, Interferon regulatory factor 3 (IFR3), Free Fatty Acid Receptor 3 (FFAR3), CD28, CD3, Interleukin Iβ (IL-1β), PAF1 Homolog, Pafl/RNA Polymerase II Complex Component (PAF1), Prostaglandin E Receptor 2 (PTGER2), or CD5.

In some embodiments, the anti-cancer transcriptional regulator decreases one or more of: Prostaglandin E Receptor 4 (PTGER4), Polyribonucleotide Nucleotidyltransferase 1(PNPT1), Interleukin 10 Receptor Subunit α (IL10Rα), or Insulin Induced Gene 1 (INSIG1).

### Immune Cells and Immune Cell Engager

In some embodiments, the immune cell comprises, or alternatively consists essentially of, or yet further consists of one or more of: an NK cell, an NKT cell, a T cell, a B cell, a dendritic cell, a splenocyte or a macrophage. In some embodiments, the immune cell comprises, or alternatively consists essentially of, or yet further consists of a splenocyte. In further embodiments, the immune cell, such as the splenocyte, specifically recognizes and binds the cancer antigen. In yet further embodiments, the immune cell, such as the splenocyte, is selected using one or more of: fluorescent activated cell sorting, magnetic-activated cell sorting, or a tetramer assay.

In some embodiments, the immune cell engager specifically recognizes and binds to the cancer antigen and a cell surface marker of the immune cell. In further embodiments, the cell surface marker of the immune cell comprises one or more of CD3, CD28, 4-1BB, CD16, NKG2D or CD64.

In some embodiments, the immune cell engager is a bispecific antibody, specifically recognizing and binding to the cancer antigen and the surface marker of the immune cell. In some embodiments, the immune cell engager comprises, or alternatively consists essentially of, or yet further consists of an antibody or an antigen-binding fragment (such as a single-chain variable fragment, i.e., an scFv) thereof specifically recognizing and binding to the cancer antigen, an antibody or an antigen-binding fragment (such as an scFv) thereof specifically recognizing and binding to the surface marker of the immune cell. In further embodiments, the antibody or antigen-binding fragment specifically recognizing and binding to the cancer antigen can be substituted with a ligand of the cancer antigen. Additionally or alternatively, the antibody or antigen-binding fragment specifically recognizing and binding to the surface marker of the immune cell can be substituted with a ligand of the surface marker. For example, an NKG2D ligand, such as MHC class I polypeptide-related sequence A (MICA), MHC class I polypeptide-related sequence B (MICB), UL16-binding protein 1 (ULBP1), UL16-binding protein 2 (ULBP2), UL16-binding protein 3 (ULBP4), UL16-binding protein 4 (ULBP4), UL16-binding protein 5 (ULBP5), UL16-binding protein 6 (ULBP6), or an equivalent of each thereof may be used to engage the immune cell expressing NKG2D.

In some embodiments, the immune cell engager is a T cell engager, optionally specifically recognizing and binding to the cancer antigen and CD3.

The following examples are included to demonstrate some embodiments of the disclosure. However, those of skill in the art should, in light of the present disclosure, appreciate that many changes can be made in the specific embodiments which are disclosed and still obtain a like or similar result without departing from the spirit and scope of the invention.

### EXPERIMENTAL METHODS

### Experimental Method No. 1-Combined OV Therapy

Two oncolytic viruses derived from C134 were constructed: C134 was engineered to express IL-12 (referred to herein as C002), while C134 was separately engineered to express EphA2 (referred to herein as C170).

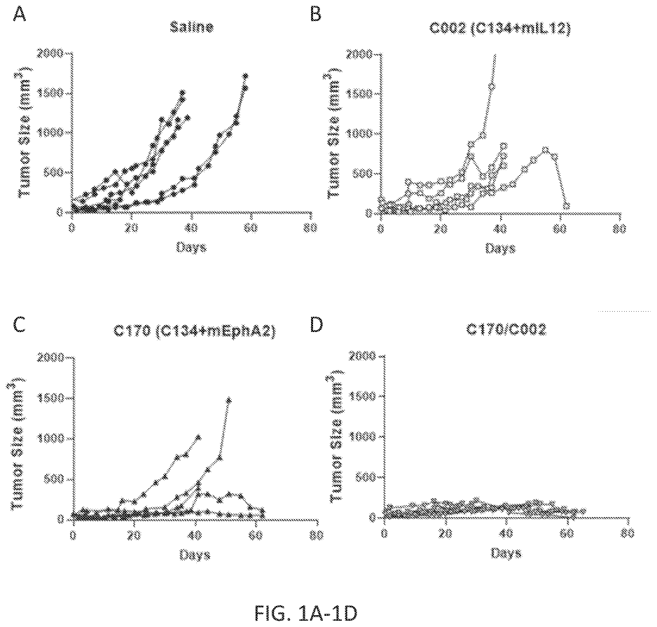

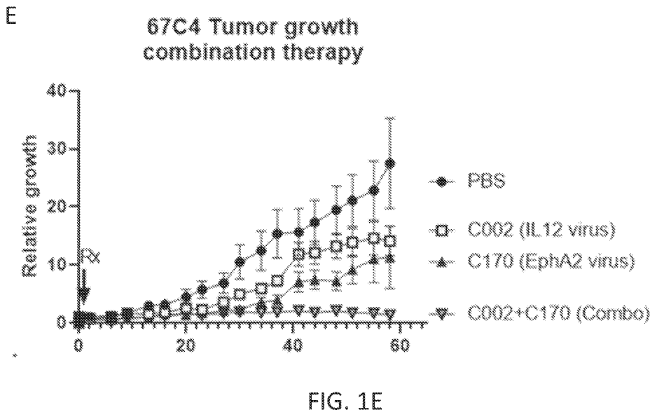

Cancer cells expressing EphA2 were implanted into mice. The mice were then divided into four groups and administered with saline/PBS (serving as a negative control), C170 alone, C002 alone, or the C170 plus C002 combination, respectively. The effectiveness of the treatments was evaluated by measuring the tumor growth over time. See, the result plotted in FIGS. 1A-1E.

All mice in the negative control group developed a tumor over 1000 mm³ and thus were sacrificed within 60 days of treatment. The individual treatments delayed the tumor growth. However, only the combination treatment suppressed the tumor growth, resulting in all mice survived the observation period (i.e., for more than 60 days post treatment). Without wishing to be bound by the theory, this result indicates that combining an oncolytic virus expressing an anti-cancer cytokine, such as IL-12, and an oncolytic virus expressing a cancer antigen, such as EphA2, leads to a more-than-additive (i.e., synergistic) effect in treating a cancer expressing the cancer antigen.

### Experimental Method No. 2-A Cytokine Delivery Approach to Improve Tumor Associated Immunotherapeutic Response

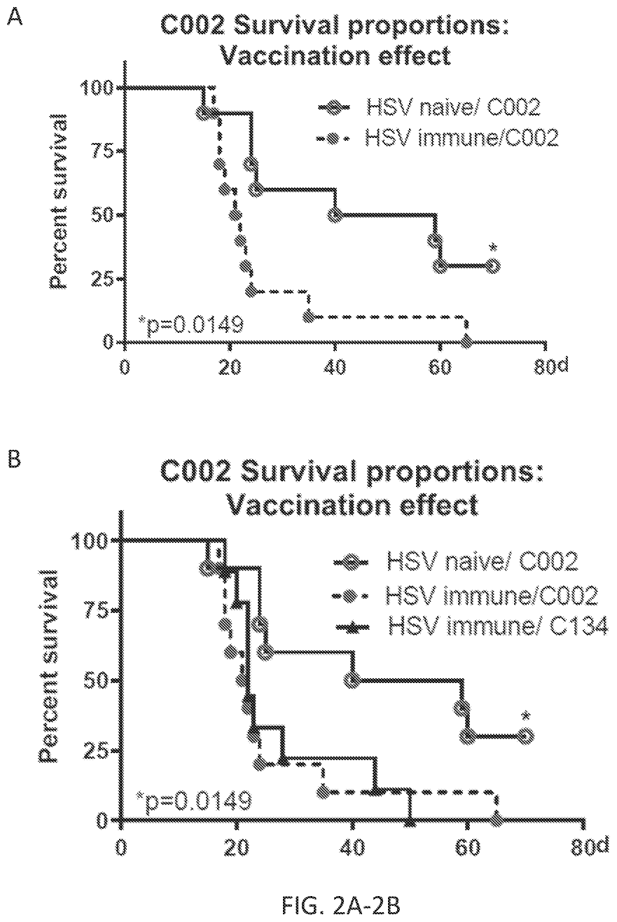

Orthotopic delayed brain tumor (DBT) based gliomas in immunocompetent syngeneic Balb/C mice were treated with 1×10⁷ PFU OV 5 day post implantation by intracorporeal (IC) injection. Naïve mice were administered with saline by intraperitoneal (IP) injection 6 and 3 weeks prior to the IC injection. The mice were immunized with 1×10⁶ ICP34.5(−) virus PFU IP 6 & 3 weeks before the IC injection, and referred to herein as the HSV immune group. The survival results are provided in FIGS. 2A-2B.

Compared to the Naïve mice, HSV immune mice had a lower survival rate (see, FIG. 2A), indicating that when repeatedly administered or if prior immunity is present, gene expressing viruses, such as C002, have shortened gene expression, and prior anti-viral immunity limits OV-based cytokine expression and therapeutic response.

Further, the HSV immune mice treated with C002 had a higher survival rate compared to the HSV immune mice treated with C0134. Without wishing to be bound by the theory, this result shows that viral based cytokine expression (e.g., IL-12) improves the treatment effects.

### Experimental Method No. 3-G207 Treatment

G207 is an attenuated replication-defective HSV-1 vector that expresses β-galactosidase following infection. It comprises the β-galactosidase gene inserted in-frame in the ribonucleotide reductase gene. As such, this recombinant virus is unable to replicate in non-dividing cells. See, for example, Yazaki et al, Cancer Res. 55(21): 4752-4756, 1995; and Mineta et al, Nature Med. 1(9): 938-943, 1995.

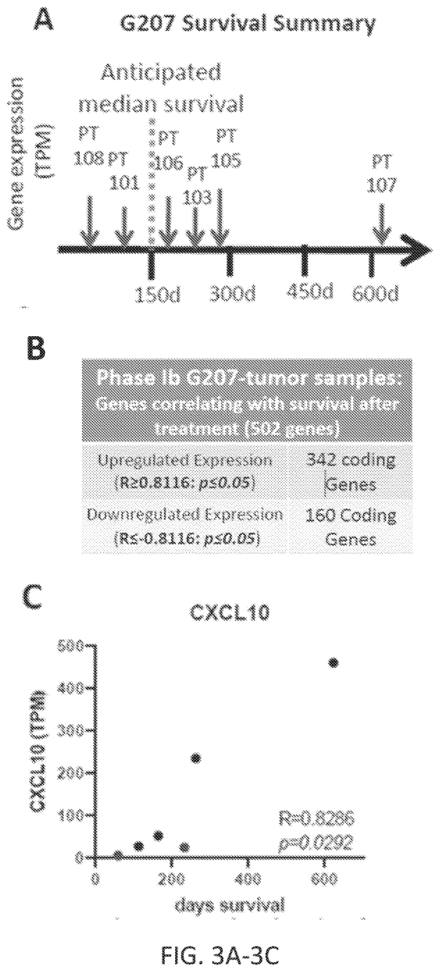

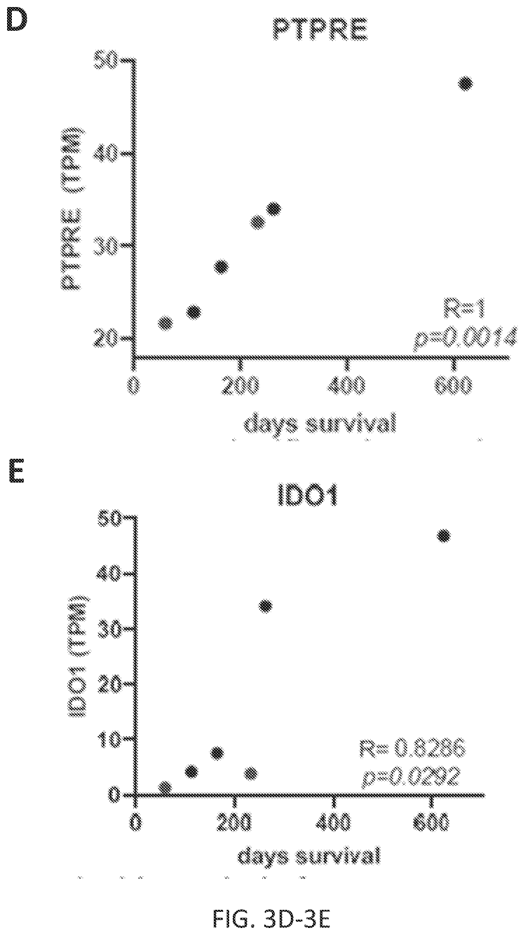

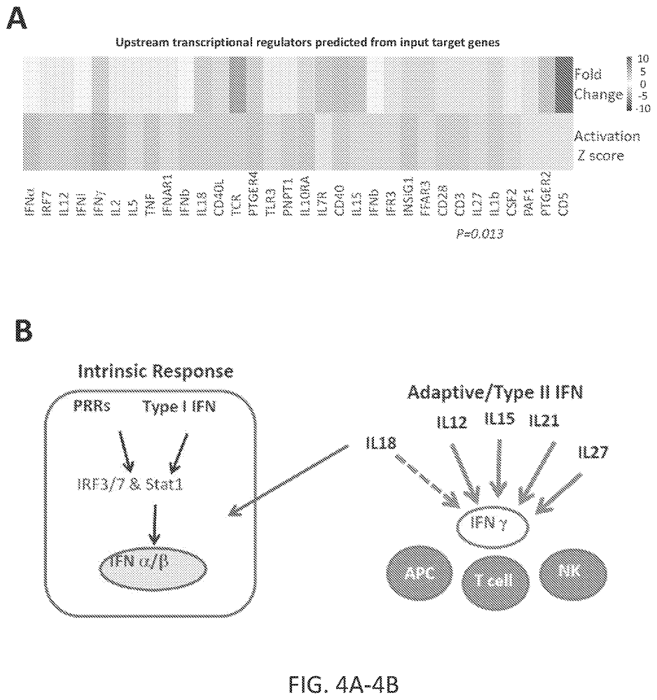

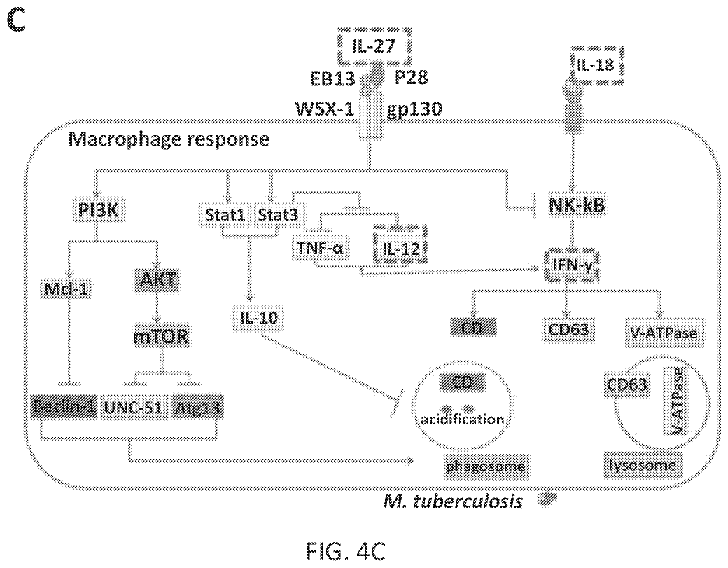

Unbiased gene expression analysis was performed from the Phase Ib G207 treated patient tumors (resected 2-5 days post G207 infusion). The results showed a direct correlation in gene expression and survival in patients for select transcripts. See, for example, FIGS. 3A-3E Further, as shown in FIG. 4, patient treated with the oncolytic HSV (oHSV, such as G207) in clinical trial who had high IL-27 levels in their treated tumors also lived longer, indicating there was a direct correlation between survival and IL-27 levels.

IL-27's role is further investigated. Oncolytic viruses expressing IL-27 is used in treating a cancer in an animal model as described herein. The effectiveness of the treatment is evaluated and compared to the oncolytic virus only treatment and optionally the IL-27 only treatment.

In some embodiments, the IL-27 treatment is further combined with an oncolytic virus expressing a cancer antigen.

### Equivalents

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

1. A composition comprising:
a first oncolytic virus expressing an anti-cancer cytokine, or an anti-cancer transcriptional regulator, or both; and
a second oncolytic virus expressing one or more of: a cancer antigen, an immune cell engager specifically recognizing and binding to the cancer antigen, or an anti-cancer cytokine that is not the one expressed by the first oncolytic virus.

2. A composition comprising:
an oncolytic virus expressing an anti-cancer cytokine, or an anti-cancer transcriptional regulator, or both; and
an immune cell specifically recognizing and binding to a cancer antigen,
with the proviso that (i) the anti-cancer cytokine does not comprise any one of interleukin 15 (IL-15), RANTES, interleukin 2 (IL-2), Tumor Necrosis Factor α (TNFα), or C-X-C Motif Chemokine Ligand 11 (CXCL11), or (ii) the immune cell does not comprise a Chimeric Antigen Receptor (CAR) T cell, or both (i) and (ii),
optionally wherein the anti-cancer cytokine comprises any one or more of: interleukin 12 (IL-12), interleukin 21 (IL-21), interleukin 27 (IL-27), or C-X-C Motif Chemokine Ligand 10 (CXCL10).

3. An oncolytic virus expressing:
an anti-cancer cytokine, or an anti-cancer transcriptional regulator, or both; and
a cancer antigen, or an immune cell engager specifically recognizing and binding to the cancer antigen, or both.

4. A composition comprising the oncolytic virus of claim 3.

5. A kit comprising:
instructions for use;
a first composition comprising a first oncolytic virus expressing an anti-cancer cytokine, or an anti-cancer transcriptional regulator; and
a second composition comprising a second oncolytic virus expressing one or more of: a cancer antigen, an immune cell engager specifically recognizing and binding the cancer antigen, or an anti-cancer cytokine that is not the one expressed by the first oncolytic virus.

6. The composition of claim 1, wherein the cancer antigen comprises an embryonic antigen.

7. The composition of claim 6, wherein the embryonic antigen comprises one or more of: Ephrin Type-A Receptor 2 (EphA2), Interleukin 13 Receptor Subunit Alpha 2 (IL 13Ra2) homodimers, carcinoembryonic antigen (CEA), or heat shock protein (hsp) gp96 (HSP96), or wherein the cancer antigen comprises or further comprises one or more of: alphafetoprotein (AFP), CA-125 (Mucin 16, Cell Surface Associated), MUC-1 (Mucin 1, Cell Surface Associated), Epithelial tumor antigen (ETA), Tyrosinase, Melanoma-associated antigen (MAGE), abnormal products of ras, p53, CD10, CD19, CD20, CD21, CD22, CD25, CD30, CD33, CD34, CD37, CD44v6, CD45, CDw52, Fms-like tyrosine kinase 3 (FLT-3, CD135), c-Kit (CD117), CSF1R (Colony Stimulating Factor 1 Receptor, CD115), CD133, PDGFR-α (CD140a), PDGFR-β (CD 140b), chondroitin sulfate proteoglycan 4 (CSPG4, melanoma-associated chondroitin sulfate proteoglycan), EGFR (Epidermal Growth Factor Receptor), de2-7-EGFR (EGFRvIII), Folate binding protein, Her2neu, Her3, PSMA (Prostate-Specific Membrane Antigen), PSCA (Prostate Stem Cell Antigen), PSA (Prostate-Specific Antigen), TAG-72, HLA-DR, IGFR, IL3R, fibroblast activating protein (FAP), Carboanhydrase IX (MN/CA IX), Carcinoembryonic antigen (CEA), EpCAM, CDCP1, Derlin1, Tenascin, frizzled 1-10, VEGFR2 (KDR/FLK1), VEGFR3 (FLT4, CD309), Endoglin, CLEC14, Tem1-8, or Tie2.

8. (canceled)

9. The composition of claim 1, wherein the anti-cancer cytokine comprises one or more of: Interleukin 12 (IL-12), Interleukin 18 (IL-18), Interleukin 15 (IL-15), Interleukin 21 (IL-21), Interleukin 27 (IL-27), C-X-C Motif Chemokine Ligand 9 (CXCL9), C-X-C Motif Chemokine Ligand 10 (CXCL10), C-X-C Motif Chemokine Ligand 11 (CXCL11), Interferon α (IFNα), Type I interferon (IFNI), Type II interferon (IFNII), Interferon γ (IFNγ), Interleukin 2 (IL-2), Interleukin 5 (IL-5), Tumor Necrosis Factor (TNF), Interferon β (IFNβ), Interleukin 18 (IL-18), CD40 Ligand (CD40L), Colony Stimulating Factor 2 (CSF2), or an equivalent of each thereof,
or wherein the anti-cancer transcriptional regulator activates one or more of: an anti-cancer cytokine, Interferon Regulatory Factor 7 (IRF7), Interferon Alpha And Beta Receptor Subunit 1 (IFNAR1), T cell Receptor (TCR), Toll-like receptor 3 (TLR3), Interleukin 7 Receptor (IL7R), CD40, Interferon regulatory factor 3 (IFR3), Free Fatty Acid Receptor 3 (FFAR3), CD28, CD3, Interleukin 1β (IL-1β), PAF1 Homolog, Paf1/RNA Polymerase II Complex Component (PAF1), Prostaglandin E Receptor 2 (PTGER2), or CD5,
or wherein the anti-cancer transcriptional regulator decreases one or more of: Prostaglandin E Receptor 4 (PTGER4), Polyribonucleotide Nucleotidyltransferase 1 (PNPT1), Interleukin 10 Receptor Subunit α (IL10Rα), or Insulin Induced Gene 1 (INSIG1), or wherein the immune cell comprises one or more of: an NK cell, an NKT cell, a T cell, a B cell, a dendritic cell, a splenocyte or a macrophage.

10-12. (canceled)

13. The composition of claim 1, wherein the immune cell comprises a splenocyte, or
wherein the immune cell engager specifically recognizing and binding to the cancer antigen and a cell surface marker of the immune cell, or
wherein the cell surface marker of the immune cell comprises one or more of CD3, CD28, 4-1BB, CD16, NKG2D or CD64, or
wherein the oncolytic virus is or is derived from adenovirus, adeno-associated virus (AAV), reovirus, measles, herpes simplex virus (HSV), Newcastle disease virus (NDV), vesicular stomatitis virus (VSV), poliovirus (PV), ECHO-7 (RIGVIR), semliki forest virus (SFV), maraba virus, coxsackievirus A21, senecavirus, a Pox virus, a retrovirus, a vesiculovirus, a parvovirus, or an influenza.

14-16. (canceled)

17. The composition of claim 13, wherein the Pox virus comprises one or more of: a vaccinia virus (VACV), a cowpox, a myxoma virus, or a racoonpox virus (RPV), squirrelpox virus, or yaba monkey tumor virus, or
wherein the HSV comprises either or both of HSV-1 or HSV-2, or
wherein the retrovirus comprises gamma-retrovirus or lentivirus, or
wherein the vesiculovirus comprises either or both of vesicular stomatitis virus or maraba virus.

18-20. (canceled)

21. The composition of claim 1, wherein the first oncolytic virus expresses IL-12 and the second oncolytic virus expresses EphA2, or
wherein the first oncolytic virus expresses IL-12 and the second oncolytic virus expresses a bispecific T-cell engager that binds to CD3 and EphA2, or
wherein the first oncolytic virus expresses IL-21 and the second oncolytic virus expresses CXCL10, or
wherein the first oncolytic virus expresses IL-21 and CXCL10, and the immune cell comprises a splenocyte that specifically recognizes and binds EphA2.

27. (canceled)

28. A method of treating a subject having a cancer, comprising administering the composition of claim 1.

29. A method of treating a subject having a cancer, comprising administering the first composition and the second composition of the kit of claim 5.

30. The method of claim 29, wherein the first composition and the second composition are administered to the subject concurrently or sequentially.

31. The method of claim 28, wherein the cancer expresses EphA2.

32-34. (canceled)
